(19) United States
(12) Patent Application Publication
(10) Pub. No.: US 2010/0167274 A1
(43) Pub. Date: Jul. 1, 2010

(54) Methods and kits for identifying nucleic acid sequences

(76) Inventors: Joel Stavans, Rechovot (IL); Dror Sagi, Rechovot (IL)

Correspondence Address: MARTIN D. MOYNIHAN d/b/a PRTSI, INC., P.O. BOX 16446, ARLINGTON, VA, 22215, US

(21) Appl. No.: 11/988,714

(22) PCT Filed: Jul. 16, 2006
(86) PCT No.: PCT/IL2006/000824

§ 371 Date: Jan. 11, 2008

Related U.S. Application Data
(60) Provisional application No. 60/699,373, filed on Jul. 15, 2005.

(51) Int. Cl. C12Q 1/68

(52) U.S. Cl. 435/6

(57) ABSTRACT

Methods and kits for detecting a nucleic acid sequence-of-interest in a sample are provided. The methods and kits use RecA-mediated strand exchange which is sensitive to a single-mismatch and which can exchange strands of non-denatured polynucleotide sequences of a sample. Thus the methods and kits of the present invention can be used for the identification of single nucleotide polymorphisms (SNPs), length of telomeric ends, numeration of chromosomes and disease-associated genes in a sample.

### FIELD AND BACKGROUND OF THE INVENTION

The present invention relates to the identification of a mismatch-sensitive RecA-catalyzed strand exchange and, more particularly, to methods and kits for detecting specific nucleic acid sequences in a sample, under denaturing or non-denaturing conditions, using a highly sensitive, RecA-mediated strand exchange which can be used for the identification of single nucleotide polymorphisms (SNPs), length of telomeric ends, numeration of chromosomes and disease-associated genes.

Homologous recombination is an essential mechanism for generating genetic diversity and rapid acquisition of novel functions. By creating new combinations out of two DNA molecules as for example during horizontal transfer in unicellular organisms or crossover between chromosomes in eukaryotes, recombination drives evolutionary adaptation and promotes speciation. Homologous recombination also plays a fundamental role during the repair and bypass of DNA lesions, enabling the resumption of DNA replication at stalled replication forks and thus the survival of the cell. In prokaryotes, recombination is carried out by the RecA protein. According to the prevailing view, the RecA-catalyzed recombination process proceeds along the following steps. First, a nucleoprotein complex is formed by the polymerization of RecA along a single-stranded DNA substrate (ssDNA), in a sequence-sensitive fashion (Bar-Ziv and Libchaber, 2001; Flory and Radding, 1982). ssDNA, which is accepted to be a prerequisite, is produced at double strand breaks by the recombination-specific helicases, RecBCD and RecG, or as a byproduct of DNA repair processes at stalled replication forks. Next, there is a search for homology between the RecA-ssDNA nucleoprotein filament and double stranded DNA (dsDNA). This search involves alignment of the nucleoprotein filament with a given tract along duplex DNA resulting in the formation of a three-stranded synaptic intermediate. Even in cases of heterology, formation of the synaptic intermediate involves unwinding of the duplex. Finally, a RecA-promoted strand exchange process between the single-stranded and double-stranded partners ensues, starting from the 3′ end of the ssDNA (Friedman-Ohana and Cohen, 1998).

Recombination is however not error-free and can proceed with a limited degree of heterology (Bazemore et al., 1997; Bucka and Stasiak, 2001; Rao and Radding, 1994), promoting genetic diversity. Moreover, genomic rearrangements such as gene duplications can arise during the SOS response, as a result of recombination of partially homologous sequences such as the rearrangement hot spots rhsA and rhsB. In E. coli, the ability of RecA to tolerate mismatches, deletions and other heterologies during recombination is held in check by mismatch repair systems (MRS). When the MRS is inactivated in the recipient organism, recombination processes between E. coli and S. typhimurium are enhanced nearly a thousand fold. In vitro, the methyl-directed MRS proteins MutS and MutL directly inhibit RecA-catalyzed strand exchange between DNA segments differing by more than 3% (Worth et al., 1994).

There are however important situations in which MRS systems are either downregulated or inactivated, thereby enhancing recombination. For example, such downregulation can be found in bacteria experiencing feast and famine lifestyles, with the latter being more the rule rather than the exception. Under famine, when E. coli cells enter stationary phase, the MutHLSU mismatch repair system is strongly downregulated. In natural E. coli isolates, 0.1-1.0% of cells are high mutators due to inactivation of mut genes. Cells may also become transient mutators through modulation of their MRS systems, via occasional transcription or translation errors. Furthermore, in bacteria other than E. coli, such as Streptococcus pneumoniae and Bacillus subtilis, MRS may play a moderate or minor role in heterology tolerance. In these situations, the determinant mechanism to ensure approximate homology must be provided by the recombination process itself. The barrier against indiscriminate recombination must be sensitive to preserve speciation for instance, but at the same time allow for an efficient process of homology search after DNA uptake.

The potential advantage of using fluorescence resonance energy transfer (FRET) was demonstrated by studying RecA-mediated strand exchange. FRET probes provide noninvasive measurement of real-time kinetics in a previously inaccessible millisecond time regime and offer great sensitivity (Gumbs and Shaner, 1998; Gupta et al., 1998). Thus, it was shown by Folta-Stogniew et al. (2004) that in RecA-catalyzed strand exchange a subset of bases exchanges at a rate that is fast enough to account for recognition of homology. Recently, the formation of a RecA-mediated double D-loop (Rice et al., 2004, Genome Res. 14:116-125; US Patent Application Publication No. 2003/0180746 to Kmiec et al.) or triple strand formation (U.S. Pat. No. 6,849,410 to Shigemori et al.) were suggested for detecting single nucleotide polymorphisms (SNPs).

Currently practiced methods of detecting nucleic acid sequences-of-interest include isolation of DNA or RNA nucleic acid sequences from the cell or tissue sample and subsequently denaturing the isolated nucleic acid sequences at high temperatures (e.g., 95° C.) to enable hybridization with labeled probes or annealing of specific primers such as for PCR amplification. Alternatively, when in situ identification of the nucleic acid sequence-of-interest is needed (e.g., in tissue biopsies or fetal cells), the sample is subjected to various denaturing agents such as alkaline solutions (e.g., sodium hydroxide) or formaldehyde, which may destroy cellular or tissue components and thus limit the use of the tissue sample for subsequent for diagnostic assays. In addition, the need of denaturing agents limits the detection methods to cells or tissue samples which are taken out of the individual in need of diagnosis and cannot be performed in vivo (i.e., within the subject). Moreover, the current in situ hybridization protocols require several hours of hybridization (e.g., at least 12 hours) which limit the efficacy of the diagnostic test for clinical applications (e.g., in case of cancer diagnosis).

Thus, there is a need, and it would be highly advantageous to develop a method of detecting a presence of a nucleic acid sequence-of-interest devoid of the above limitations.

### SUMMARY OF THE INVENTION

According to one aspect of the present invention there is provided a method of detecting a nucleic acid sequence-of-interest in a sample, the method comprising: (a) providing a complex of a polynucleotide sequence of the sample and a recombinase; (b) incubating the complex with a labeled polynucleotide which comprises a nucleic acid sequence complementary to the nucleic acid sequence-of-interest under conditions suitable for exchange between the polynucleotide sequence and the labeled polynucleotide; and (c) measuring a rate and/or amount of the exchange to thereby detect the nucleic acid sequence-of-interest in the sample.

According to another aspect of the present invention there is provided a kit for detecting a nucleic acid sequence-of-interest in a sample, the kit comprising packaging materials and at least one agent identified by the packaging material as being suitable for measuring a rate and/or amount of a recombinase-mediated exchange between a polynucleotide sequence of the sample and a labeled polynucleotide which comprises a nucleic acid sequence complementary to the nucleic acid sequence-of-interest.

According to yet another aspect of the present invention there is provided a method of detecting a nucleic acid sequence-of-interest in a sample, the method comprising: (a) providing a complex of a labeled polynucleotide which comprises a nucleic acid sequence complementary to the nucleic acid sequence-of-interest and a recombinase and; (b) incubating the complex with the sample under conditions suitable for exchange between the labeled polynucleotide and the polynucleotide sequence of the sample; and (c) measuring a rate and/or amount of the exchange to thereby detect the nucleic acid sequence-of-interest in the sample.

According to still another aspect of the present invention there is provided a kit for detecting a nucleic acid sequence-of-interest in a sample, the kit comprising packaging materials and at least one agent identified by the packaging material as being suitable for measuring a rate and/or amount of a recombinase-mediated exchange between a labeled polynucleotide which comprises a nucleic acid sequence complementary to the nucleic acid sequence-of-interest and a polynucleotide sequence of the sample.

According to further features in preferred embodiments of the invention described below, the nucleic acid sequence-of-interest comprises a single nucleotide polymorphism (SNP).

According to still further features in the described preferred embodiments the recombinase is selected from the group consisting of RecA, Rad51, DMC1, Sin, Cre, RadA and Rec12.

According to still further features in the described preferred embodiments the polynucleotide sequence is a PCR product.

According to still further features in the described preferred embodiments the polynucleotide sequence of the sample is a non-denatured fragment of a genomic nucleic acid sequence.

According to still further features in the described preferred embodiments measuring of the rate and/or the amount of the exchange is effected in situ.

According to still further features in the described preferred embodiments the polynucleotide sequence of the sample is a single strand DNA (ssDNA).

According to still further features in the described preferred embodiments the polynucleotide sequence of the sample is a double strand DNA (dsDNA). According to still further features in the described preferred embodiments detecting is effected in situ.

According to still further features in the described preferred embodiments incubating is effected for a time period selected from the range of 1-20 minutes.

According to still further features in the described preferred embodiments the conditions comprise hydrolysable ATP.

According to still further features in the described preferred embodiments the labeled polynucleotide which comprises the nucleic acid sequence complementary to the nucleic acid sequence-of-interest is a double stranded DNA (dsDNA) and whereas one strand of the dsDNA is labeled with a fluorescent acceptor and a second strand of the dsDNA is labeled with a fluorescent donor.

According to still further features in the described preferred embodiments the labeled polynucleotide which comprises the nucleic acid sequence complementary to the nucleic acid sequence-of-interest is a dsDNA and whereas one strand of the dsDNA is labeled at a 5′-end and a second strand of the dsDNA is labeled at a 3′-end.

According to still further features in the described preferred embodiments the fluorescent acceptor and the fluorescent donor are positioned on the dsDNA such that an average physical distance therebetween is selected from the range of 30-60 Angstrom.

According to still further features in the described preferred embodiments the at least one agent is a hydrolysable ATP.

According to still further features in the described preferred embodiments the labeled polynucleotide which comprises the nucleic acid sequence complementary to the nucleic acid sequence-of-interest is labeled on one strand and whereas the polynucleotide sequence of the sample is labeled on one strand.

According to still further features in the described preferred embodiments the labeled polynucleotide which comprises the nucleic acid sequence complementary to the nucleic acid sequence-of-interest is a plurality of labeled polynucleotides and whereas the plurality of labeled polynucleotides are configured as an array.

According to still further features in the described preferred embodiments the labeled polynucleotide which comprises the nucleic acid sequence complementary to the nucleic acid sequence-of-interest is attached to a solid support.

According to still further features in the described preferred embodiments the nucleic acid sequence-of-interest is selected from the group consisting of a repeated nucleic acid sequence, a disease-associated nucleic acid sequence and/or a genomic fragment of a chromosome.

According to still further features in the described preferred embodiments the method and/or the kit are for detecting a presence of the SNP, telomeric instability, and/or DNA or chromosomal aberrations.

According to still further features in the described preferred embodiments the nucleic acid sequence-of-interest comprises at least 25 nucleic acids.

According to still further features in the described preferred embodiments the conditions comprise non-denaturing conditions.

The present invention successfully addresses the shortcomings of the presently known configurations by providing a sensitive and robust method of detecting nucleic acid sequence-of-interest which can be used for detection of SNPs, length of telomeric ends, disease-associated genes and chromosomal numeration.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c are schematic illustrations depicting RecA-induced strand exchange using FRET-labeled probes. FIG. 1a illustrates recombination between a single strand DNA (ssDNA) and a double stranded DNA (dsDNA). A ssDNA substrate covered with RecA (rectangles) is combined with dsDNA labeled at both strands on the 5′ and 3′ ends with a donor (d)-acceptor (a) pair. Note the relative lengths of all oligomers. After strand exchange, no energy transfer takes place and only fluorescence from the donor is detected. FIG. 1b illustrates ds-DNA-dsDNA recombination. FIG. 1c illustrates ssDNA-dsDNA recombination in the presence of competing duplexes exhibiting only partial homology (over part of their length) with the dsDNA.

FIGS. 2a-f depict recombination efficiency (strand exchange) as a function of the number of mismatches and their respective location on the labeled dsDNA. FIGS. 2a-b are scatter plots depicting ssDNA-dsDNA strand exchange as a transfer efficiency (TE; FIG. 2a) or the fraction of exchanged oligonucleotides (FIG. 2b). The sequences of ssDNA oligonucleotides are set forth by SEQ ID NO:1 (black, 100% identical to the sequence of the labeled dsDNA), SEQ ID NO:2 (orange; a single mismatch sufficiently far away from the oligonucleotide ends), SEQ ID NO:3 (green, two mismatches), SEQ ID NO:4 (red, three mismatches), SEQ ID NO:5 (blue, four mismatches). The mismatch nucleotides in each ssDNA are shown in red and consist of G-C exchanges relative to the dsDNA sequence. All experiments were carried out in the presence of 2 mM ATP and with increasing concentrations of RecA (0-3 μM). Note the ˜10% difference in the fraction of exchanged strands observed using the single mismatch oligonucleotide (SEQ NO:2, orange) as compared with the identical ssDNA (SEQ ID NO:1, black). Also note that the presence of more mismatches results in lower efficiency of strand exchange. FIGS. 2c-d are scatter plots depicting the effect of mismatch location on ssDNA-dsDNA strand exchange. The fraction of exchange was measured as function of RecA concentration. FIG. 2c illustrates the effect of the number and location of mismatches on strand exchange. The sequences of the ssDNA oligonucleotides are set forth by SEQ ID NO:6 (black, 100% identical to the sequence of the labeled dsDNA), SEQ ID NO:7 (blue, empty circles; one mismatch at the third nucleotide of from the 3′-end), SEQ ID NO:8 (blue, full circles; ±5 one mismatch at the fifth nucleotide of from the 3′-end), SEQ ID NO:9 (green, empty circles; two mismatches at nucleotide position 5 and 7 from the 3′-end), SEQ ID NO:3 (green, full circles, two mismatches), SEQ ID NO:10 (red, empty circles; three mismatches) and SEQ ID NO:4 (red, full circles; three mismatches). FIG. 2d illustrates the effect of one mismatch close to the 5′-end on strand exchange. The sequences of the ssDNA oligonucleotides are set forth by SEQ ID NO:12 (blue, empty circle; a single mismatch on the fifth nucleotide from the 5′-end) and SEQ ID NO:11 (blue, filled circles; 100% identical to the sequence of the labeled dsDNA). FIG. 2e is a scatter plot depicting the effect of energy dissipation in recombination: fraction of strand exchange as function of RecA concentration, in the presence (full symbols) or absence (empty symbols) of 2 mM ATP, or in the presence of 0.2 mM ATPγS (crosses). The experiments were carried out with an oligonucleotide having a sequence which is identical to that of the labeled dsDNA (blue; SEQ ID NO:1), and with an oligonucleotide having including two mismatches (with respect to the labeled dsDNA) near the 3′-end (red; SEQ ID NO:9). The black circle denotes the result of adding 2 mM ATP to the sample with 2.5 μM RecA but without ATP. Note the low fraction of exchange in the absence of ATP or in the presence of ATPγS as compared with the efficient exchange obtained when ATP is present (blue and red full circles) or added (black circle). FIG. 2f is a graph depicting fluorescence from the donor as a function of time in the experiments described in FIG. 2e in the presence of ATP (red) or ATPγS (blue), at 2 mM and 0.2 mM respectively. Note the high and stable fluorescence observed in the presence of ATP as compared with the low and fluctuating signal observed in the presence of ATPγS, indicating oligonucleotide aggregation and possible failure of strand exchange.

FIG. 3 is a graph depicting the effect of double-stranded competitor exhibiting partial homology on the rate of strand exchange between fully homologous ssDNA and dsDNA. In the experiments depicted in FIGS. 3 and 4 the competitor concentration was tenfold that of the labeled dsDNA. Fraction of labeled duplexes undergoing strand exchange as a function time in the presence of competitor duplexes bearing homology of 0 (full circles), 15 (empty circles) and 35 (full triangles) base pairs (bp). Full lines are exponential fits to the data.

FIG. 4 is a graph depicting the rise time of the curves in FIG. 3 as function of the extent of homology of the competitor duplex. Note that in the presence of sequences with, partial homology the recombination rate between the ssDNA-dsDNA is retarded.

FIGS. 5a-b depict the effect of a single mismatch located close to the 5′-end of the invading polynucleotide on the fraction of strand exchange. FIG. 5a—The sequences of the target duplex (SEQ ID NOs:19 and 20), and the two invading strands (SEQ ID NOs:6 and 18) are shown. FIG. 5b—a scatter plot depicting the fraction of exchange as a function of RecA concentration in the presence of a perfectly matched invading strand (empty circles; SEQ ID NO:6) or a mismatched invading strand (fill circles; SEQ ID NO:18). Note the significant difference in the fraction of strand exchange at all RecA concentrations in the range of 0.25 μM to 2.5 μM.

FIG. 6 depicts one embodiment of the present invention. The target duplex (SEQ ID NOs:19 and 26) is labeled only at one strand (SEQ ID NO:19) with a donor fluorophore (TAMRA), while the other strand (SEQ ID NO:26) is unlabeled. Each of the invading strands (SEQ ID NO:27, which is 100% complementary to SEQ ID NO:19 and SEQ ID NO:28, which includes one mismatch at position 5 with respect to SEQ ID NO:27) is labeled with an acceptor (Cy5). Following strand exchange the formed duplex includes two fluorophores (TAMRA and Cy5), whose FRET signal can be measured.

FIGS. 7a-b depict one embodiment of the present invention in which RecA-mediated exchange is performed using a labeled double-stranded probe containing a overhang and an unlabeled double stranded target duplex. FIG. 7a—A sequence diagram of the labeled probe (invader double-stranded DNA) shown in FIG. 7b which is composed of a 69-mer oligonucleotide (SEQ ID NO:29) labeled at the 5′ with Cy5 and a 31-mer oligonucleotide (SEQ ID NO:30) labeled at the 3′ with TAMRA. The bolded nucleotides represent a staggered sequence. The nucleotides marked in yellow, green, light blue, grey and purple represent the 1^{st}, 6^{th}, 7^{th}, 8^{th} and 11^{th} positions, respectively, from the 5′-end of the TAMRA-labeled oligonucleotide (SEQ ID NO:30), and are further referred to in the description of FIG. 8 hereinbelow. FIG. 7b—A schematic illustration of the Rec-A exchange reaction between a labeled double-stranded probe containing a relatively large overhang of 31-mer which polymerizes with RecA and two unlabeled double-stranded target duplexes. The two unlabeled target duplexes represent two double-stranded polynucleotides (which include a nucleic acid sequence-of-interest) which are present in a cell of an individual. The two polynucleotides can be identical to each other (i.e., with no SNP with respect to each other) or can vary from each other in at least one nucleotide (i.e., the SNP nucleotide). The position of the putative SNP is marked with “n” and a double arrow. Each of the double-stranded polynucleotides represents a perfect duplex (no mismatches between the two strands forming the double strand DNA). RecA polymerizes with the 31-mer single strand portion of the acceptor-conjugated strand (SEQ ID NO:29), such that following interaction with the target duplex, the acceptor-conjugated strand exchanges one of the strands of the target duplex, while the other strand can form double stranded duplex at a portion of its sequence with the displaced donor-conjugated strand. Alternatively, the displaced donor-conjugated strand can be released into the solution.

FIG. 8 is a bar graph depicting the effect of the position of a single mismatch with respect to the 5′-end of the invader strand on the fraction of exchange with a non-denatured target duplex. Invader and target duplexes are as depicted in FIGS. 7a-b, hereinabove. Full exchange values (i.e., a value of “1”) were determined by measuring the fluorescence at 580 nm (donor) and 670 nm (acceptor) of 0.1 μM labeled probe (SEQ ID NOs:29-30) in the presence of ×50 excess of unlabeled target duplex (SEQ ID NOs:31-32; i.e., 5 μM) which was denatured at 95° C. and cooled down to room temperature for 5 hours. The exchange values of the probe in the presence of 0.2 μM of the non-denatured target duplexes were measured and are presented as a fraction of the full exchange. The blue column represents the fraction of exchange of probe in the presence of a non-denatured target duplex (SEQ ID NOs:31-32) which exhibits full homology to the labeled probe. Note that while a single mismatch in the first 5 bases forming the staggered end of the invader strands (such a present in the target duplex formed by the oligonucleotides set forth by SEQ ID NOs:33-34) has a limited effect (e.g., a decrease of 4% in the fraction of exchange), a more significant effect is observed when the mismatch is located at nucleotide 6 (i.e., the first nucleotide of the duplex, an effect of about 10% using a target duplex composed of SEQ ID NOs:35-36) or even more significantly when the mismatch is located at nucleotides 7, 8 or 11 (e.g., 20%; using target duplexes composed of SEQ ID NOs:37-38, 39-40 or 41-42, respectively).

FIG. 9 depicts one embodiment of the present invention. The invader strands are labeled with a donor and an acceptor and include a repetitive sequence [5′-TTAGGG (SEQ ID NO:43)] such as that present in the telomeric end of a chromosome. The target duplex is unlabeled and represents a non-denatured duplex of genomic DNA from the telomeric end of a chromosome. RecA polymerizes with the single stranded portion of the invader strands. Following interaction with the target duplex, the acceptor-conjugated strand exchanges one strand of the target duplex and forms double stranded duplex with the genomic DNA. The fraction of exchange is indicative of the number of repeats of the repetitive sequence.

### DESCRIPTION OF THE PREFERRED EMBODIMENTS

The present invention is of methods and kits for detecting a nucleic acid sequence-of-interest in a sample, and more particularly, the present invention is of the use of RecA-catalyzed strand exchange for the identification of single nucleotide polymorphisms (SNPs), length of telomeric ends, numeration of chromosomes and disease-associated genes in a sample.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Homologous recombination is an essential mechanism for generating genetic diversity and rapid acquisition of novel functions. In prokaryotes, the recombination process is RecA-catalyzed and consists of the following steps. First, a nucleoprotein complex is formed by the polymerization of RecA along a single-stranded DNA substrate (ssDNA), in a sequence-sensitive fashion (Bar-Ziv and Libchaber, 2001; Flory and Radding, 1982). Next, there is a search for homology between the RecA-ssDNA nucleoprotein filament and a double stranded DNA (dsDNA). This search involves alignment of the nucleoprotein filament with a given tract along the duplex DNA resulting in the formation of a three-stranded synaptic intermediate. Finally, a RecA-promoted strand exchange process occurs between the single-stranded and double-stranded partners, starting from the 3′ end of the ssDNA (Friedman-Ohana and Cohen, 1998).

The potential advantage of using fluorescence resonance energy transfer (FRET) was demonstrated by studying RecA-mediated strand exchange. FRET probes provide noninvasive measurement of real-time kinetics in a previously inaccessible millisecond time regime and offer great sensitivity (Gumbs and Shaner, 1998; Gupta et al., 1998). Recently, the formation of a RecA-mediated double D-loop (Rice et al., 2004, Genome Res. 14:116-125; US Pat. Appl. No. 20030180746 to Kmiec et al., which is fully incorporated herein by reference) or triple strand formation (U.S. Pat. No. 6,849,410 to Shigemori et al., which is fully incorporated herein by reference) were suggested for detecting single nucleotide polymorphisms (SNPs). These methods are based on the use of ATP-γS for the formation of D-loop or triple strand and the inhibition of strand exchange. In addition, to avoid interaction with RecA, the annealing oligonucleotides used by Rice et al., (2004, Supra) were heavily modified at their backbone [e.g., peptide nucleic acids (PNAs) with acetyl groups, locked nucleic acids (LNAs; Proligo), or 2′-O-Methyl-RNA].

Currently practiced methods of detecting nucleic acid sequences-of-interest include isolation of DNA or RNA nucleic acid sequences from the cell or tissue sample and subsequently denaturing the isolated nucleic acid sequences at high temperatures (e.g., 95° C.) to enable hybridization with labeled probes or annealing of specific primers such as for PCR amplification. Alternatively, when in situ identification of the nucleic acid sequence-of-interest is desired (e.g., in tissue biopsies or fetal cells), the sample is subjected to various denaturing agents such as alkaline solutions (e.g., sodium hydroxide) or formaldehyde, which may destroy cellular or tissue components and thus limit the use of the sample for subsequent diagnostic assays. In addition, the need of denaturing agents limits the detection methods to cells or tissue samples which are taken out of the individual in need of and cannot be performed in vivo. Moreover, the current in situ hybridization protocols require several hours of hybridization (e.g., at least 12 hours) which limit the efficiency of the diagnostic test for clinical applications (e.g., such as in bed-side procedures).

While reducing the present invention to practice, the present inventors have uncovered that RecA-mediated strand exchange is sensitive to single mismatches and thus can be used as a highly sensitive method for detecting nucleic acid sequences-of-interest which comprise SNPs. This is in sharp contrast to the other studies described hereinabove in which inhibition of strand exchange rather than strand exchange occurrence is indicative of a presence or an absence of a SNP.

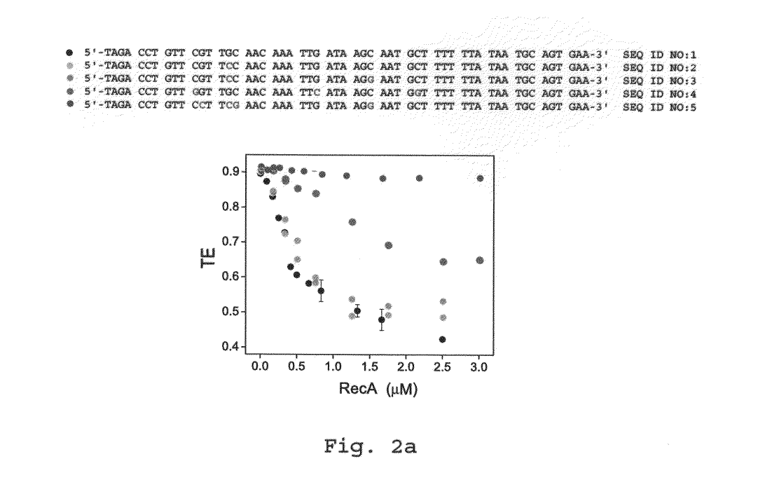

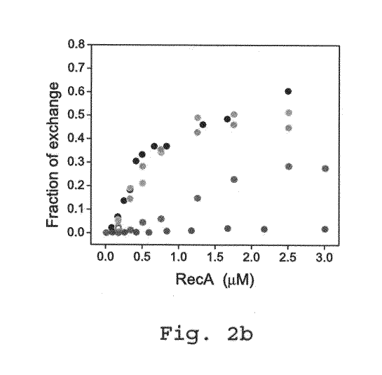

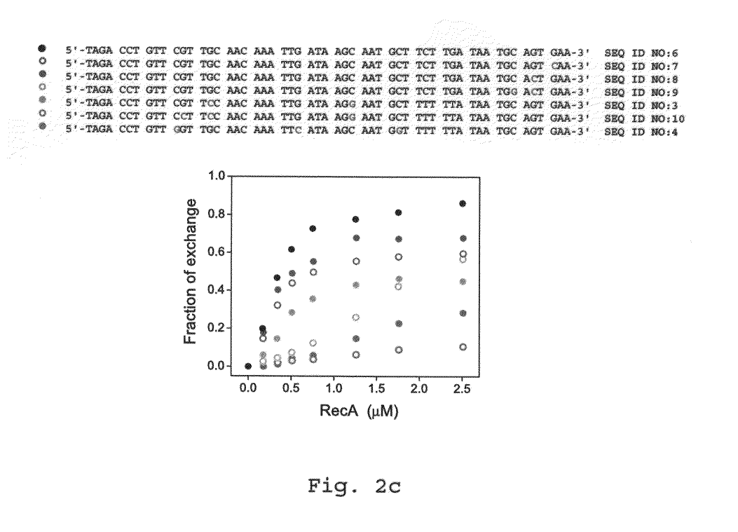

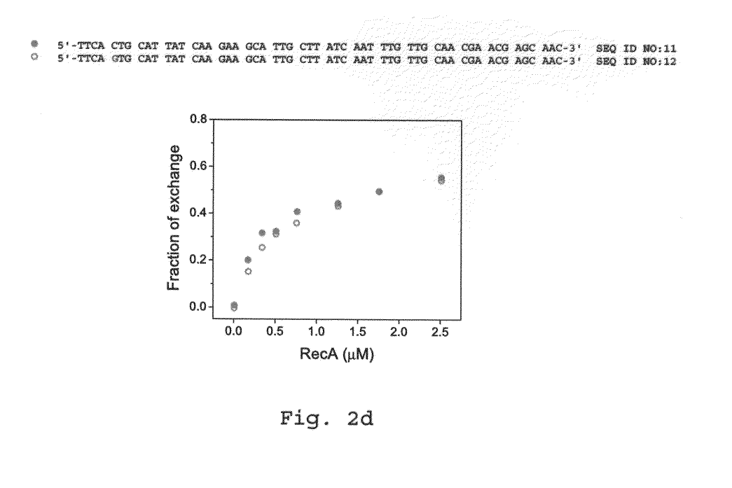

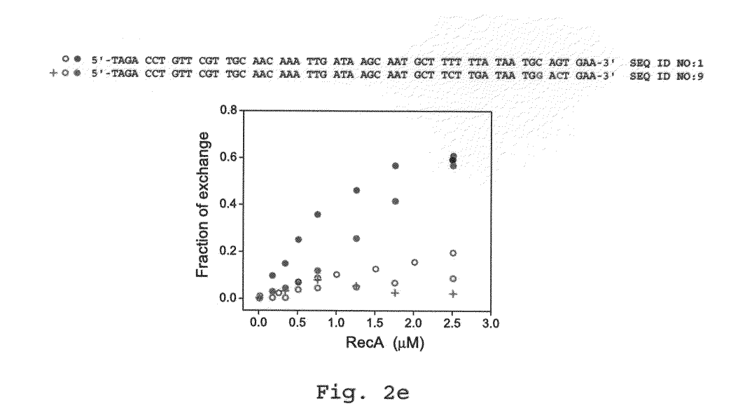

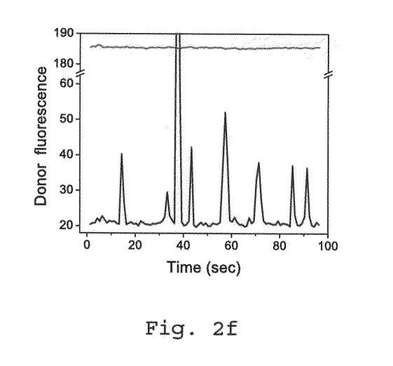

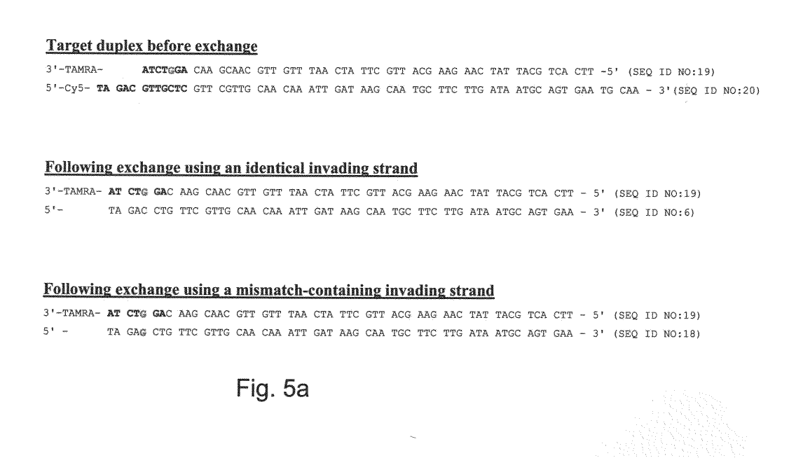

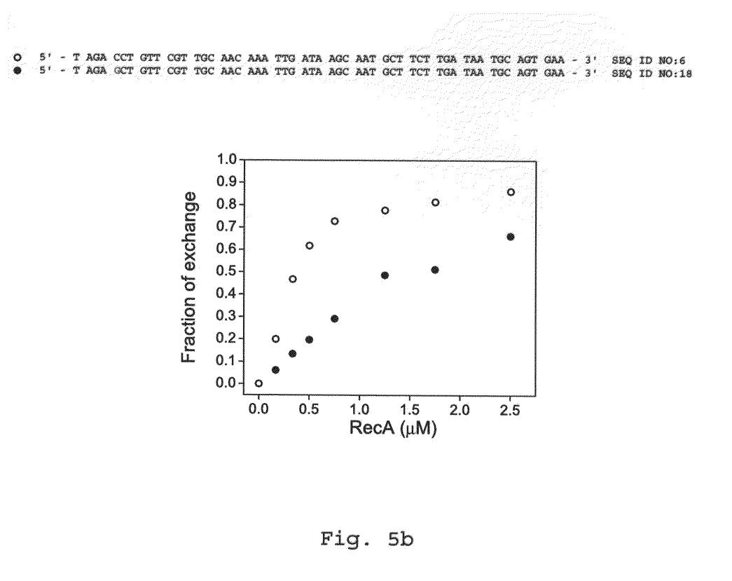

As is shown in FIGS. 2a-d and 5a-b, the present inventors have uncovered that RecA-mediated recombination is dependent on both the number and location of the mismatches. In addition, as is shown in FIGS. 5a-b, the discrimination of mismatches by RecA is more efficient when the mismatches correspond to the staggered end of the target dsDNA (e.g., close to the 5′-end of the invading ssDNA as set forth by SEQ ID NO:18) than to the other end of the duplex.

In addition, while further reducing the present invention to practice, the present inventors have uncovered that RecA-mediated strand exchange can be effected using a non-denatured double strand DNA such as the DNA duplexes naturally present in cells and tissues and thus can be used for rapid and sensitive detection of nucleic acid sequences-of-interest in a sample.

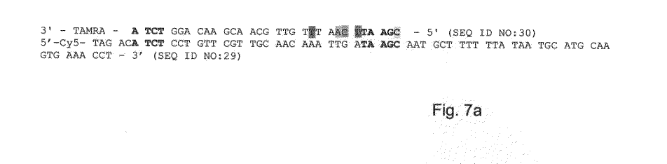

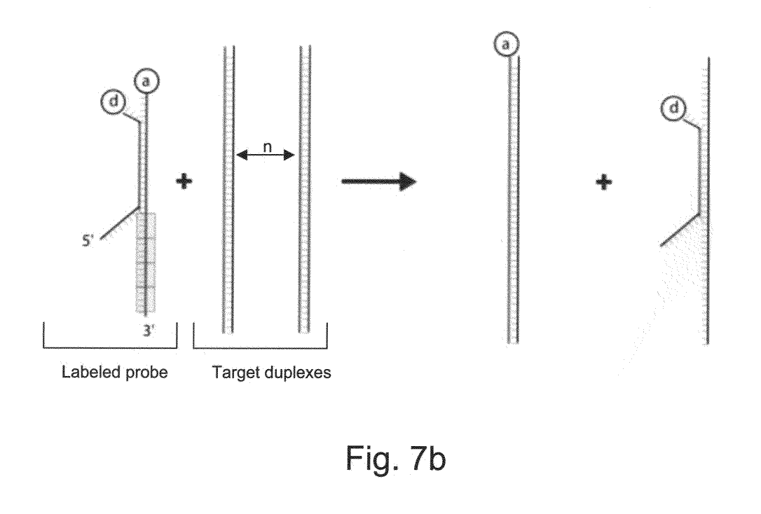

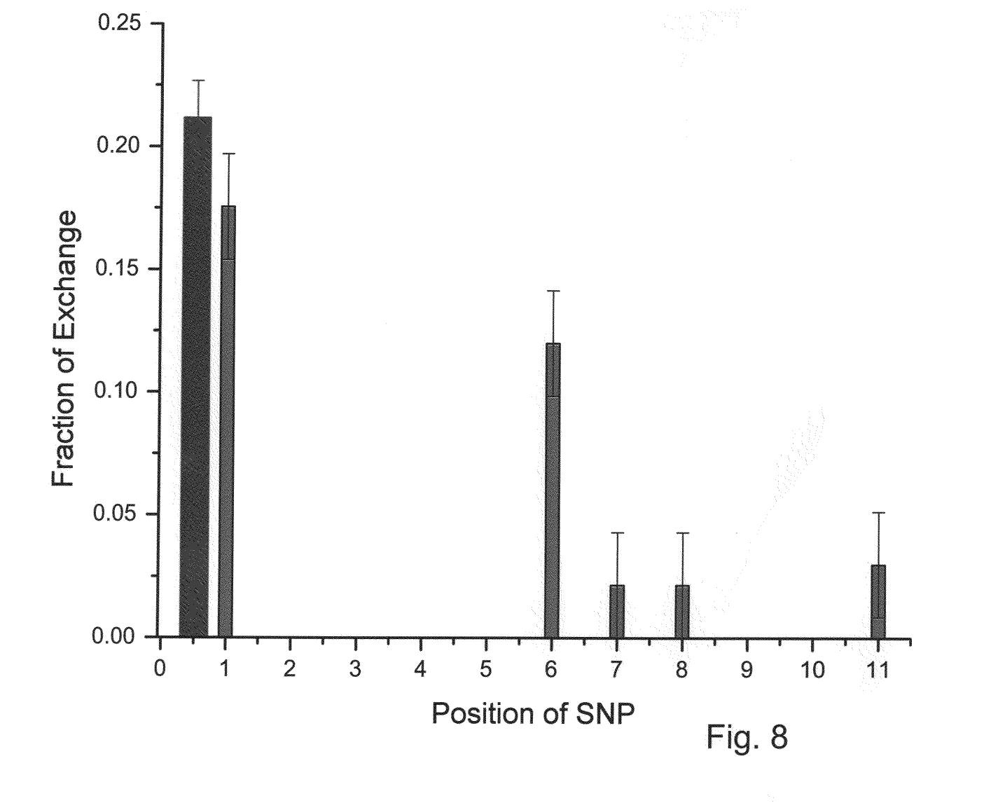

As is shown in FIGS. 7a-b and 8 and is described in Example 4 of the Examples section which follows, RecA-mediated exchange can occur in the presence of a non-denatured duplex such as the dsDNA present in cells. Moreover, as is further shown in FIG. 8, the fraction of exchange is dependent on the position of a mismatch between the exchanged strands. Thus, while a mismatch located within the staggered end has a moderate effect on the fraction of exchange (e.g., 4%), a mismatch located in a nucleotide within the duplex has a significant effect on the fraction of exchange (e.g., 10-20%). Thus, these results suggest the use of a RecA-mediated exchange for a fast detection (e.g., within minutes instead of hours) of a nucleic acid sequence-of-interest in a double stranded DNA as naturally present in a cell or tissue sample without subjecting the sample to denaturing conditions. Such a nucleic acid sequence-of-interest can be a SNP, a disease-associated nucleic acid sequence, a repetitive nucleic acid sequence and/or a mutated nucleic acid sequence.

Thus, according to one aspect of the present invention there is provided a method of detecting a nucleic acid sequence-of-interest in a sample. The method is effected by: (a) providing a complex of a polynucleotide sequence of the sample and a recombinase; (b) incubating the complex with a labeled polynucleotide which comprises a nucleic acid sequence complementary to the nucleic acid sequence-of-interest under conditions suitable for exchange between the polynucleotide sequence and the labeled polynucleotide; and (c) measuring a rate and/or amount of the exchange to thereby detect the nucleic acid sequence-of-interest in the sample.

The phrase “nucleic acid sequence-of-interest” as used herein refers to any nucleic acid sequence which detection in the sample of the present invention is desired. Such a nucleic acid sequence can be for example a nucleic acid sequence which comprises a single nucleotide polymorphism (SNP), a disease-associated nucleic acid sequence [e.g., a nucleic acid sequence of a virus such as Hepatitis virus or human immunodeficiency virus (HIV)], a mutated nucleic acid sequence such as a nucleic acid sequence formed following inversion, rearrangement, insertion, alternative splicing and the like, a repetitive nucleic acid sequence such as that present in the telomeres of the chromosomes and for which the length of the repetitive sequence is indicative for the presence of cancerous cells, fetal cells, and/or a histocompatibility complex nucleic acid sequence (e.g., HLA, which can be used for determining tissue compatibility for tissue transplantation). Additionally, the nucleic acid sequence of the present invention can be any nucleic acid sequence-of-interest which detection is desired for research purposes.

The length of the nucleic acid sequence-of-interest can be from a few nucleotides (e.g., 10 nucleic acids) to about 500 nucleic acids. Preferably, the nucleic acid sequence-of-interest is at least 20 nucleic acids in length, more preferably, at least 25 nucleic acids, at least 30 nucleic acids at least 35 nucleic acids, at least 40 nucleic acids, at least 45 nucleic acids in length.

The term “polymorphism”, as used herein, refers to a condition in which two or more different nucleotide sequences can exist at a particular locus in DNA. The phrase “single nucleotide polymorphism (SNP)” refers to any nucleic acid change (substitution), deletion, insertion, inversion and/or duplication of one or more nucleotides in a polynucleotide sequence. Non-limiting examples of nucleic acid substitutions include an A→G substitution (R type SNP), a C→T substitution (Y type SNP), an A→T substitution, an A→C substitution, a G→T substitution and a G→C (S type SNP). It will be appreciated that such a substitution, deletion, insertion, inversion and/or duplication can be benign (i.e., with no effect on the regulation, level and/or activity of a gene or a gene product) or may lead to an abnormal regulation, expression and/or activity of a gene or a gene product and thus can be related to a pathological condition or a disease (e.g., a disease-causing-mutation such as a missense mutation, a non-sense mutation, a splice mutation, a mutation in a regulatory sequence such as a promoter mutation and the like).

The sample used by the method according to this aspect of the present invention can be a sample made of in vitro constituents (e.g., which includes synthetic nucleic acid sequences, as long as they are amenable to recombinase activity, including PCR products which do not comprise synthetic components) or can be any biological sample known in the art which contains naturally occurring nucleic acid sequences, such as plasma, serum, spinal fluid, lymph fluid, the external sections of the skin, respiratory, intestinal, and genitourinary tracts, tears, saliva, sputum, milk, blood cells, tumors, neuronal tissue, organs, samples of in vivo cell culture constituents and/or cells of a subject (i.e., which are present in vivo, in the subject). In addition, the sample can be derived from any cell of a conceptus (i.e., an embryo, a fetus or an extraembryonic membrane of an ongoing pregnancy as well as of a terminated pregnancy) such as a blood cell, an amniotic cell, an extraembryonic membrane cell and/or a trophoblast cell can be used.

The method according to this aspect of the present invention is effected by: (a) providing a complex of a polynucleotide sequence of the sample and a recombinase; (b) incubating the complex with a labeled polynucleotide which comprises a nucleic acid sequence complementary to the nucleic acid sequence-of-interest under conditions suitable for exchange between the polynucleotide sequence and the labeled polynucleotide; and (c) measuring a rate and/or amount of the exchange to thereby detect the nucleic acid sequence-of-interest in the sample.

The polynucleotide sequence of the sample of the present invention can be any synthetic or naturally occurring nucleic acid sequence in the form of a complementary deoxyribonucleic acid (cDNA), a genomic DNA, cellular RNA (e.g., hnRNA, mRNA, tRNA, rRNA), a polymerase chain reaction (PCR) product, an RT-PCR product and/or a composite polynucleotide sequence (e.g., a combination of the above). Preferably, the polynucleotide sequence of the sample of the present invention is DNA (e.g., genomic DNA, PCR product and/or RT-PCR product).

According to one preferred embodiment of the present invention the polynucleotide sequence of the sample of the present invention is a single strand (ss) DNA or a double strand (ds) DNA which may be produced by PCR or multiplex PCR. As used herein, the phrase “multiplex PCR” refers to a PCR reaction performed with more than one pair of PCR primers and thus results in several PCR products. Methods of employing multiplex PCR are further described hereinbelow.

As used herein the phrase “a labeled polynucleotide which comprises a nucleic acid sequence complementary to the nucleic acid sequence-of-interest” refers to a labeled synthetic or naturally occurring double stranded or single stranded nucleic acid sequence of which one strand of the double stranded nucleic acid sequence or the single stranded nucleic acid sequence comprises a nucleic acid sequence that is complementary (i.e., in terms of nucleic acid complementation via hydrogen bond formation) to the nucleic acid sequence-of-interest described hereinabove. For example, while a synthetic single stranded nucleic acid sequence can be a single stranded oligonucleotide, a synthetic double stranded nucleic acid sequence can be formed of two complementary ssDNA molecules such as oligonucleotides (see for example, the oligonucleotides set forth by SEQ ID NOs:19 and 20 which form the dsDNA presented in FIG. 5a). Alternatively, a naturally occurring single stranded nucleic acid sequence can be a genomic fragment (e.g., a result of digestion with a restriction enzyme, PCR, RT-PCR) which is further denatured (e.g., by heating at 95° C.) to two single stranded strands. On the other hand, a naturally occurring double stranded nucleic acid sequence can be a non-denatured genomic fragment. Labeling methods and/or labeling configuration of the labeled polynucleotide of the present invention are further described hereinbelow.

The term “oligonucleotide” refers to a single stranded or double stranded oligomer or polymer of deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions. Methods of producing oligonucleotides are further described hereinbelow.

The oligonucleotides of the present invention (e.g., the labeled polynucleotide described hereinabove) can be generated according to any oligonucleotide synthesis method known in the art such as enzymatic synthesis, liquid phase or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC. Liquid phase synthesis of oligonucleotides can be performed using methods known in the art (see for example, Bonora G M, et al., 1998, Biol. Proced. Online. 1: 59-69; Padiya K J and Salunkhe M M., 2000, Bioorg. Med. Chem. 8: 337-42). It will be appreciated that for the preparation of multiple labeled polynucleotides, a large scale oligonucleotide synthesis can be utilized essentially as described elsewhere (Anderson N G. et al., Appl Biochem Biotechnol. 1995 July-September; 54(1-3):19-42; Rahmann S., Proc IEEE Comput Soc Bioinform Conf. 2002; 1:54-63).

The length of the labeled polynucleotide of the present invention can vary from a few nucleotides (e.g., 10 nucleotides) to long nucleic acid sequences (e.g., a couple of hundred nucleotides or even longer). Typically, the length of the labeled polynucleotide is at least 20, more preferably, at least 25, more preferably, at least 30, more preferably, at least 35, more preferably, at least 40, more preferably, at least 45, more preferably, at least 50 nucleotides, more preferably, at least 55, more preferably, at least 60 nucleotides, more preferably, at least 65, more preferably, at least 70 nucleotides, more preferably, at least 75, more preferably, in the range of 60-200 nucleotides, more preferably, at least 100 nucleotides, even more preferably, at least 120 nucleotides.

It will be appreciated that in case a genomic DNA is used to generate the labeled polynucleotide of the present invention, such a DNA can be cleaved to the desired length using e.g., an endonuclease. On the other hand, if a PCR product or an RT-PCR product is utilized such products can be prepared of a desired length using specific PCR primers.

As used herein, the term “recombinase” refers to any prokaryotic or eukaryotic enzyme capable of DNA recombination. Non-limiting examples of recombinase proteins which can be used along with the present invention include RecA (e.g., GenBank Accession No. PO₃₀₁₇; SEQ ID NO:25), Rad51 (GenBank Accession No. NP002866.2; SEQ ID NO:22), DMC1 (GenBank Accession No. NP_{—}008999), Sin (GenBank Accession No. NP_{—}395551), Cre (GenBank Accession No. YP_{—}006472), RadA (GenBank Accession No. NP_{—}341799) and Rec12 (GenBank Accession No. NP_{—}593479).

As is mentioned before, the recombinase used by the present invention is capable of discriminating between two polynucleotides having a single mismatch, such that the rate and/or amount of strand exchange is significantly slower in the presence of a mismatch. In addition, as described in Example 4 of the Examples section which follows, the recombinase used by the present invention is also capable of exchanging a labeled polynucleotide with one strand of a polynucleotide sequence of a duplex DNA (i.e., a non-denatured dsDNA).

The recombinase of the present invention can be purchased from a variety of commercial vendors such as Sigma (RecA from Escherichia coli strain GE 1171/pGE22 Cat. No. R7272), or New England Biolabs (Cat. No. MO249L), purified from e.g., bacteria (such as E. coli) or mammalian cells or recombinantly produced from host cells using methods known in the art (see for example, Weistock G M, et al., 1979, Proc. Natl. Acad. Sci. 76: 126-130). Recombinant recombinase thus produced can be qualified by testing exchange activity which is SNP sensitive as described in details in the Examples section which follows.

According to presently preferred configurations, the recombinase used by the method of the present invention is RecA.

RecA has been cloned from many prokaryotic and eukaryotic organisms such as E. Coli (RecA; GenBank Accession No. PO₃₀₁₇; SEQ ID NO:25) and Homo sapiens [RAD51 (RecA homologue); GenBank Accession No. NP_{—}002866.2; SEQ ID NO:22) and the coding sequences of RecA or its homologues are available via various databases including the NCBI (www.ncbi.nlm.nih.gov/).

It will be appreciated that the present invention can also utilize a variety of recombinase proteins (from a variety of organisms and/or species) or functional homologues thereof which exhibit the desired activity (i.e., DNA recombination). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to SEQ ID NO:24 (GenBank Accession No. V00328), as determined using the BestFit software of the Wisconsin sequence analysis package, utilizing the Smith and Waterman algorithm, where gap weight equals 50, length weight equals 3, average match equals 10 and average mismatch equals −9.

It will be appreciated that a recombinase homologue (e.g., recombinase mutant) in which strand exchange is more sensitive to the presence of mismatches is highly suitable for use by the method of the present invention. Such mismatch-sensitive mutants can be identified by screening phage-display libraries using a functional assay (i.e., the capacity of strand exchange in the presence of a mismatch) as described in the Examples section which follows (e.g., a FRET labeled polynucleotide duplex and a non-labeled invading strand).

Additionally or alternatively, a recombinase homologue can be artificially modified in order to include the desired properties. Modification of polypeptides (e.g. polypeptides with a catalytic activity such as enzymes) can be effected using numerous protein directed evolution technologies known in the art [for review see Kuchner and Arnold (1997) TIBTECH 15:523-530]. Typically, directed enzyme evolution begins with the creation of a library of mutated genes. Gene products that show improvement with respect to the desired property or set of properties are identified by selection or screening, and the gene(s) encoding those enzymes are subjected to further cycles of mutation and screening in-order to accumulate beneficial mutations. This evolution can involve few or many generations, depending on the progress observed in each generation.

Preferably, for successful directed evolution a number of requirements are met; the functional expression of the enzyme in a suitable microbial host; the availability of a screen (or selection) sensitive to the desired properties; and the identification of a workable evolution strategy.

Examples of mutagenesis methods which can be used in enzyme directed evolution according to this aspect of the present invention include but are not limited to UV irradiation, chemical mutagenesis, poisoned nucleotides, mutator strains [Liao (1986) Proc. Natl. Acad. Sci. U.S.A 83:576-80], error prone PCR [Chen (1993) Proc. Natl. Acad. Sci. U.S.A 90:5618-5622], DNA shuffling [Stemmer (1994) Nature 370:389-91], cassette [Strausberg (1995) Biotechnology 13:669-73], and a combination thereof [Moore (1996) Nat. Biotechnol. 14:458-467; Moore (1997) J. Mol. Biol. 272:336-347].

Screening and selection methods are well known in the art [for review see Zhao and Arnold (1997) Curr. Opin. Struct. Biol. 7:480-485; Hilvert and Kast (1997) Curr. Opin. Struct. Biol. 7:470-479]. Typically, selections are attractive for searching larger libraries of variants, but are difficult to device for enzymes that are not critical to the survival of the host organism. Further more, organisms may evade imposed selective pressure by unexpected mechanisms. Less stringent functional complementation can be useful in identifying variants which retain biological activity in libraries generated using relatively high mutagenic rates [Suzuki (1996) Mol. Diversity. 2:111-118; Shafikhani (1997) Biol. Techniques 23:304-310; Zhao and Arnold (1997) Curr. Opin. Struct. Biol. 7:480-485].

To express an exogenous recombinase (e.g., RecA or RAD51, including a recombinase homologue as described hereinabove) in host cells (e.g., mammalian, bacteria, plant, yeast), a polynucleotide sequence encoding a recombinase [e.g., the bacterial RecA (GenBank Accession No. V00328; SEQ ID NO:24) or homo sapiens RAD51 (GenBank Accession No. NM_{—}002875; SEQ ID NO:22)] is preferably ligated into a nucleic acid construct suitable for host cell expression. Such a nucleic acid construct includes a promoter sequence for directing transcription of the polynucleotide sequence in the cell in a constitutive or inducible manner. Constitutive promoters suitable for use with the present invention are promoter sequences which are active under most environmental conditions and most types of cells such as the cytomegalovirus (CMV) and Rous sarcoma virus (RSV).

The nucleic acid construct (also referred to herein as an “expression vector”) of the present invention includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator, a polyadenylation signal, and other specialized elements (such as viral replicons) intended to increase the level of expression of cloned nucleic acids or to facilitate the identification of cells that carry the recombinant DNA.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide. In addition, the expression vector of the present invention can also include sequences engineered to enhance stability, production, secretion (e.g., a secretion signal), purification, yield or toxicity of the expressed peptide [see e.g., Booth et al. (1988) Immunol. Lett. 19:65-70; and Gardella et al., (1990) J. Biol. Chem. 265:15854-15859].

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that a variety of prokaryotic or eukaryotic cells can be used as host-expression systems to express the recombinase of the present invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of the present invention.

Recovery of the recombinant polypeptide is effected following an appropriate time in culture. The phrase “recovering the recombinant polypeptide” refers to collecting the whole fermentation medium containing the polypeptide and need not imply additional steps of separation or purification. Not withstanding the above, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

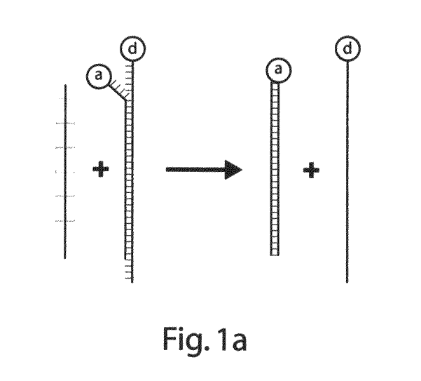

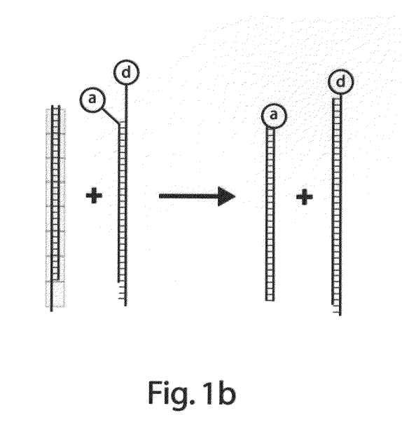

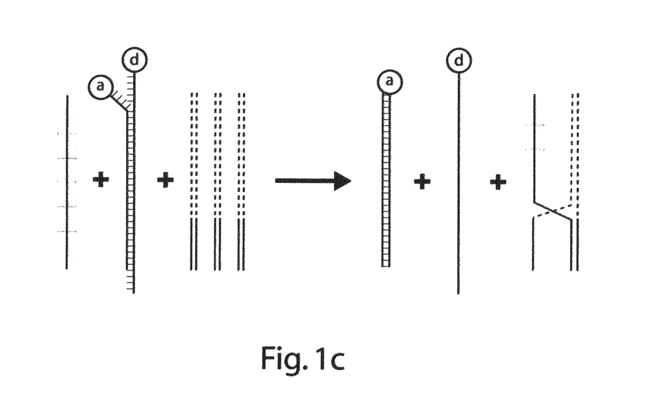

According to this aspect of the present invention, the polynucleotide sequence of the present invention forms a complex with the recombinase. As used herein, the term “complex” refers to the non-covalent association between the polynucleotide sequence of the present invention (e.g., a PCR product) and the recombinase enzyme (i.e., a nucleoprotein complex). Examples of such complexes can be found in FIGS. 1a and b.

Various methods can be used to label the polynucleotide of the present invention (i.e., the polynucleotide which comprises a nucleic acid sequence complementary to the nucleic acid sequence-of-interest). These include fluorescent labeling with a fluorophore conjugated via a linker or a chemical bond to at least one nucleotide, or the use of a covalently conjugated enzyme (e.g., Horse Radish Peroxidase) and a covalently conjugated substrate (e.g., o-phenylenediamine) which upon interaction therebetween yields a colorimetric or fluorescent color. Alternatively, the displaced oligonucleotide of the duplex, which does not switch partners, can be radiolabeled, and the reaction of exchange can be followed using a gel shift assay.

As used herein the term “fluorophore” refers to any entity which can be excited by light to emit fluorescence. Such a fluorphore can be an artificial or a naturally occurring molecule (e.g., fluorescein, Texas Red, rhodamine), or a quantum dot. Quantum dots are coated nanocrystals fabricated from semiconductor materials in which the emission spectrum is controlled by the nanocrystal size. Quantum dots have a wide absorption spectrum, allowing simultaneous emission of fluorescence of various colors with a single excitation source. Quantum dots can be modified with large number of small molecules and linker groups such as conjugation of amino (PEG) or carboxyl quantum dots to streptavidin (Quantum Dot Corporation, Hayward, Calif., USA).

Thus, the labeled polynucleotide of the present invention (e.g., which comprises a nucleic acid sequence complementary to the SNP) can be labeled at any nucleotide. Preferably, labeling occurs on at least one end of the polynucleotide, i.e., the 3′ or the 5′-end. It will be appreciated that labeling can be effected on one strand only (e.g., at the 5′-end), or can be effected on both strands of the dsDNA (e.g., one strand can be labeled on the 5′-end and the other strand can be labeled on the 3′-end).

It will be appreciated that the polynucleotide sequence of the sample (e.g., a genomic DNA in which the nucleic acid sequence-of-interest, e.g., a SNP is detected) can be also labeled with a fluorophore, conjugated to a non-fluorophore FRET donor or acceptor conjugated to an enzyme, conjugated to a substrate or remain unlabeled and/or unconjugated. It will be appreciated that if the polynucleotide sequence of the sample is a dsDNA, only one strand of the dsDNA is preferably labeled with a fluorophore or conjugated as described hereinabove.

Preferably, the method of the present invention uses fluorescently labeled nucleotides, i.e., nucleotides which are directly or indirectly conjugated to fluorophores such as fluorescein and its derivatives (e.g., fluorescein isothiocyanate), rhodamine and its derivatives, dansyl, umbelliferone, Texas red, and quantum dots (e.g., EviTags available from Evident Technologies New York (http://www.evidenttech.com)]. See for example, Wang J, et al., 2005, Nucleic Acids Res. 33(2):e23; Mayall F, et al., 2003, J. Clin. Pathol. 56(10): 728-30, which are fully incorporated herein by reference. For example, as is described under General Materials and Experimental Methods of the Examples section which follows, the present inventors used oligonucleotides which were labeled at their 5′ or 3′-ends with either Tetramethylrhodamine (TAMRA) or Cy5 fluorescent groups (Thermo Bioscience GmbH, Ulm, Germany) and were HPLC and PAGE purified. Preferably, the fluorophores used by the present invention are suitable for FRET analysis. During FRET, energy is transferred non-radiatively through a dipole-dipole interaction between two fluorescent molecules, a donor and an acceptor. This transfer occurs without the emission of fluorescence from an excited state of the donor, produced by light whose wavelength is within the excitation spectrum of the donor, and the acceptor, provided donor and acceptor are in proximity. The excited state of the acceptor thus produced decays by fluorescence, through the emission of a photon whose wavelength is within the emission spectrum of the acceptor.

According to this aspect of the present invention the complex formed between the recombinase and the polynucleotide sequence of the sample (in this configuration also called the “invading polynucleotide”) is incubated with the labeled polynucleotide of the present invention (i.e., which comprises the nucleic acid sequence complementary to the nucleic acid sequence-of-interest; in this configuration also called the “target duplex”) under conditions suitable for exchange between the polynucleotide sequence of the sample and the labeled polynucleotide. Such incubation is effected for a time period which enables such an exchange. According to preferred embodiments of the present invention such a time period can be between one second to a few minutes, more preferably, between 10 seconds to 30 minutes, more preferably, between 30 seconds to 25 minutes, more preferably, between 30 seconds to 20 minutes, more preferably, between 30 seconds to 20 minutes, more preferably, between 30 seconds to 15 minutes, more preferably, between 1 minute to 10 minutes, more preferably, between 2-8 minutes, more preferably, between 2-6 minutes, more preferably, between 2-5 minutes, even more preferably, about 5 minutes.

As is shown in FIGS. 2a-d and is described in Example 1 of the Examples section which follows, the exchange reactions of the present invention were carried out in the presence of 2 mM ATP. On the other hand, when a non-hydrolysable ATP was utilized (e.g., ATPγS), or if ATP was omitted from the reaction (i.e., in the absence of ATP), no significant exchange was observed (FIG. 2e, Example 2 of the Examples section which follows).

Thus, according to preferred embodiments of the present invention, the conditions used by the method of the present invention and which enable the exchange reaction include a hydrolysable ATP which is necessary for strand exchange in DNA recombination. As used herein, the phrase “hydrolysable ATP” refers to the adenosine triphosphate molecule which is capable of undergoing an energy releasing reaction of hydrolysis to adenosine diphosphate (ADP) and phosphate (Pᵢ).

In addition, such conditions also include specific temperatures and buffers which are suitable for DNA strand exchange. The temperature can be in the range of 20-70° C. depending on the recombinase used (e.g., RecA, RAD51, thermally stable RecA-like protein). It will be appreciated, that in case the recombinase used is derived from a thermally stable bacteria, such as Thermus thermophilus, the exchange reaction can take place at high temperatures (in the range of 50-70° C.). Typically, the exchange reaction takes place at a temperature in the range of 30-40° C., more preferably, at 37° C. (i.e., a physiological temperature).

The buffer used for the exchange reaction can be any water-based buffer (e.g., Hepes-KOH, Tris, Triethanoleamine-HCL, Pipes) and at a range of pH values from 7 to 7.6, for example, pH 7.5. Such buffer may include additional salts such MgCl₂ (e.g., at a concentration of 20 mM) and other additives [e.g., DTT or bovine serum albumin (BSA)].

The rate and/or amount of exchange between the polynucleotide sequence of the sample and the labeled polynucleotide of the present invention can be measured using various approaches depending on the mode of labeling (e.g., fluorescent FRET probes or enzyme-substrate interactions) and the labeling configuration (i.e., if the labeled polynucleotide is labeled on both strands or only one, if the polynucleotide of the sample is labeled or not, or if the labeled polynucleotide is a single stranded polynucleotide labeled with a fluorophore).

In case FRET probes are used (i.e., the polynucleotides are labeled with fluorophores suitable for FRET analysis), measuring the rate and/or amount of exchange can be done by quenching of the donor fluorescence alone (e.g., in case a non-fluorophore acceptor is used), the emission of fluorescence characteristic of the acceptor alone (e.g., in case a fluorophore acceptor is used), or both (in case a fluorophore acceptor is used). It will be appreciated that the donor and acceptor can be of the same fluorophore [in this case FRET is measured by fluorescent polarization (see for example, Chen X, 2003, Comb. Chem. High. Throughput Screen. 6(3): 213-23; Luo et al., 2004, Acta Biochim. Biophys. Sin. (Shanghai). 36(6): 379-84)] or of different fluorophores (in this case FRET is measured by the appearance of fluorescence of the acceptor and/or by quenching of donor fluorescence). As is described under Materials and Experimental Methods in the Examples section which follows, both ensemble and single pair (sp)-FRET measurements can be performed using confocal fluorescence detection, and laser light illumination (Argon 514 nm line). Briefly, for FRET measurement the following set up can be used: The laser beam is collimated, reflected with a dichroic mirror (Omega Optical, DRLP540), and focused by an objective (Zeiss 100×, NA 1.4, oil immersion). The sample is placed inside a glass chamber, with a cover-glass window facing the objective. To detect freely diffusing molecules and reduce background fluorescence, the focal point is placed about 10 μm from the glass surface. Fluorescence is collected with the same objective, and focused on a 100 μm pinhole, after residual laser light is filtered with a 514 nm optical notch-filter (HNPF-514.5-1.0, Kaiser Optical Systems). Following the pinhole, the light is divided with a second dichroic mirror (Omega Optical, DRLP630), and focused on two avalanche photodiodes (Perkin Elmer, SPCM AQR-14) (one for the donor and the other one for the acceptor). To increase the signal-to-background ratio optical band-pass filters can be used in the donor channel (e.g., Omega Optical, 580DF30), and in the acceptor channel (e.g., Omega Optical, 670DF40). Detection is performed with a counter based on a PC counting board (National Instruments, DAQ 6602). For ensemble measurements a concentration of 90 nM DNA is used. To avoid photobleaching the sample is illuminated with 14 μW laser power. The intensities from both donor and acceptor channels are used to calculate transfer efficiency (TE) as described previously (Sagi, D., et al., 2004, J. Mol. Biol. 341, 419-428).

The TE is the ratio of the acceptor over the sum of donor and acceptor channels, after subtracting from the acceptor channel the laser's direct excitation and the overlap between donor and acceptor. In these experiments, a decrease of donor fluorescence of 100 counts results in an increase in acceptor yield of 5-6 counts. Therefore, the factor γ, which measures the quantum yield, is estimated to be 0.05. Due to the configuration of the donor and acceptor in the labeled duplex, the donor's emission increases up to nine fold in a strand displacement assay.

The fraction of exchange is extracted from donor and acceptor intensities as follows: first, the intensities from donor (d1) and acceptor (al) channels are measured in a sample containing hybridized labeled duplexes. Next, the intensities from donor (d2) and acceptor (a2) channels are measured in a sample containing unhybridized, fully separated donor-labeled and acceptor-labeled ssDNA oligonucleotides, at the same concentration as in the duplex measurements. To ensure their separation during this measurement, the labeled ssDNAs are first hybridized with complementary unlabeled ssDNA partner strands. For a given RecA concentration, the assumption is that labeled ssDNA species are partitioned into doubly-labeled duplexes, or singly-labeled ones resulting from strand exchange. The fraction of doubly-labeled dsDNA undergoing strand exchange X is then calculated from the equations:

d=X·d2+(1−X)·d1

a=X·a2+(1−X)·a1

where “d” and “a” are the measured intensities of the donor and acceptor respectively.

According to one preferred embodiment of this aspect of the present invention, the labeled polynucleotide is labeled on both strands such that one strand is labeled at the 3′-end and the other strand is labeled at the 5′-end. Preferably, one strand of the labeled polynucleotide is labeled with a fluorescent acceptor and the second strand of the labeled polynucleotide is labeled with a fluorescent donor.

As is mentioned before, FRET is based on a certain distance which enables energy transfer between the donor and the acceptor. Such a distance is usually in the range of 10-100 Angstrom. According to preferred embodiments of the present invention the distance between the donor and the acceptor is 30-60 Angstrom. More preferably, such a distance is in the range of 35-55 Angstrom, even more preferably, in the range of 40-50 Angstrom. To achieve such a distance the target duplex is preferably designed with staggered ends at the side of the duplex bearing the donor-acceptor pair. For example, to create a labeled polynucleotide with a staggered end the oligonucleotides forming the labeled polynucleotide exhibit sequence complementarity for about 20-120 nucleic acids followed by a sequence of non-complementary nucleic acids of about 5-25 nucleic acids. It will be appreciated that the length of non-complementary sequence can be different in each of the strands forming the target duplex. For example, one oligonucleotide can include 5 nucleic acids of non-complementary sequence and the other can include 15 nucleic acids of non-complementary sequence (see for example the oligonucleotides set forth by SEQ ID NOs:19 and 20 which form the target duplex as depicted in FIG. 5a). This configuration enables a near maximum transfer efficiency by avoiding short-distance effects in resonant energy transfer essentially as described elsewhere (Schuler, B., et al., 2005, Proc Natl Acad Sci USA 102, 2754-2759).

According to another preferred embodiment of this aspect of the present invention Molecular Beacons are utilized to follow RecA-mediated strand exchange. For example, one strand of the target duplex is covalently attached to a fluorophore while the other strand of the labeled polynucleotide is covalently attached to a quencher. Thus, prior to the addition of RecA and the polynucleotide sequence of the sample, the labeled polynucleotide does not fluoresce (due to the presence of the quencher in a close vicinity to the fluorophore). On the other hand, following strand exchange and dissociation of the exchanged strand which is labeled with the quencher, the fluorescence of the strand labeled with the fluorophore can be detected. It will be appreciated that for this configuration the labeled end of the labeled polynucleotide can be designed as a complete double strand or may include a staggered end as described hereinabove.

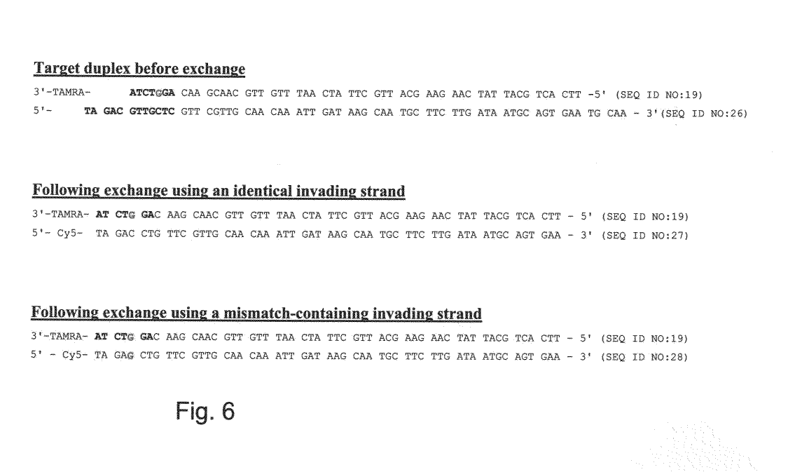

According to another preferred embodiment of this aspect of the present invention, the labeled polynucleotide is labeled at only one strand of the duplex (e.g., at the 5′- or 3′-end) with either a donor or an acceptor. In this configuration the polynucleotide sequence of the sample which forms a complex with the recombinase is also labeled with either a donor or an acceptor. Thus, if the labeled polynucleotide duplex is labeled with a donor, then the polynucleotide sequence of the sample which forms a complex with the recombinase is labeled with an acceptor, and vice versa. It will be appreciated that in case the polynucleotide forming the complex with recombinase is a dsDNA, the labeled strand is the strand complementary to the labeled strand of the labeled polynucleotide duplex (a non-limiting example of such configuration is depicted in FIG. 6). Using any of these configurations which involve a labeled polynucleotide sequence of the sample, strand exchange is followed by either the quenching of the donor (the labeled polynucleotide of the present invention which comprises a nucleic acid sequence complementary to the nucleic acid sequence-of-interest) or the emission of the acceptor (the labeled polynucleotide sequence of the sample which forms a complex with RecA).

According to another preferred embodiment of this aspect of the present invention, the labeled polynucleotide is a single stranded polynucleotide which is labeled at one end (e.g., at the 5′- or 3′-end) with a fluorophore. Following strand exchange the fluorescence signal of the fluorophore can be detected by any fluorescent reader and/or a fluorescent microscope according to methods well known in the art. It will be appreciated that in this configuration, the unlabeled duplex (e.g., target genomic DNA) is preferably attached to a solid support (e.g., a glass which includes a cell or tissue specimen) and following RecA-mediated strand exchange the excess of labeled polynucleotide is washed away (using known wash procedures such as those practiced for FISH), and the only detectable fluorescent signal results from the labeled polynucleotide which formed a duplex with one strand of the target DNA.

In case the enzyme-substrate approach is utilized for labeling the labeled polynucleotide of the present invention (i.e., which comprises a nucleic acid sequence complementary to the nucleic acid sequence-of-interest), the rate and/or amount of exchange can be measured by following the enzymatic reaction between the enzyme and the substrate. For example, if the conjugated enzyme is horse radish peroxidase (HRP) and the substrate is chlorpromazine (CPZ), then the exchange reaction can be followed by measuring nitroblue tetrazolium (NBT) reduction and O₂ consumption. Alternatively, the substrate can be conjugated to a fluorophore which following interaction with the enzyme is released from the substrate and thus can be detected.

Preferably, the nucleic acid sequence-of-interest includes one allele of a SNP along with the flanking sequence. In this case one strand of the labeled polynucleotide is complementary to one strand of nucleic acid sequence of one allele of the SNP. For example, if the SNP is a substitution and the DNA flanking sequence including the SNP is 5′-TTCACTGCATTATCAAGAAGCA TTGCTTATCAATTTGTTGCAACGAACAG[C/G]TCTA-3′ (SEQ ID NO:21; the “[G/C]” corresponds to the polymorphic nucleotide) then one strand of nucleic acid sequence of one allele of the SNP can be of the following sequence: 5′-TTCACTGCATTATCAAGAAGCATTGCTTATCAATTTGTTGCAACGAACAGGTCTA-3′ (SEQ ID NO:19; the underlined G corresponds to the G allele of the SNP). In this case, the labeled polynucleotide will include one strand of the following complementary sequence: 5′-TAGACCT GTT CGTTG CAA CAA ATT GAT AAG CAA TGC TTC TTG ATA ATGC AGT GAA-3′ (SEQ ID NO:45).

Thus, according to this aspect of the present invention the measured rate and/or amount of exchange is indicative of the presence of the nucleic acid sequence-of-interest in the sample (e.g., the presence or absence of the SNP). In case one strand of the polynucleotide sequence of the sample [e.g., one of the invading strand(s) in this configuration] is identical (e.g., absence of SNP) to one strand of the labeled polynucleotide (e.g., target duplex in this configuration) the rate and/or amount of exchange would be fast (e.g., more than 80% of the labeled polynucleotide molecules are exchanged within 1-5 minutes). On the other hand, if one strand of the polynucleotide sequence of the sample exhibits at least one mismatch as compared to one strand of the labeled polynucleotide (e.g., presence of SNP) the rate and/or amount of exchange is low (e.g., less than 30% of the labeled polynucleotide molecules are exchanged within 1-5 minutes). It will be appreciated that in case the sample includes a mixture of two polynucleotide sequences representing two alleles of the same SNP (e.g., in a DNA sample derived from a heterozygote individual), the rate of exchange would have an intermediate value (e.g., 50-60% of exchange within 1-5 minutes) between the fast and slow rates described hereinabove.

Preferably, to determine the presence of a nucleic acid sequence-of-interest (e.g., a SNP) in a polynucleotide sequence of a sample (for which the presence or absence or the nucleic acid sequence-of-interest is unknown, i.e., a test sample) the rate of exchange between the polynucleotide sequence of the test sample and the labeled polynucleotide of the present invention is compared to the rate of exchange between a polynucleotide sequence of a control DNA sample which comprises a nucleic acid sequence that is identical to the nucleic acid sequence of the exchanged strand of the labeled polynucleotide. For example, as is shown in FIGS. 5a and b, the rate of exchange between the polynucleotide set forth by SEQ ID NO:18 (the invading strand of the test sample) is compared to the rate of exchange of the polynucleotide set forth by SEQ ID NO:6 (the control DNA sample), which is identical to the exchanged strand of the target duplex (SEQ ID NO:20).

As is mentioned hereinabove, the present inventors have uncovered that RecA strand exchange can be also used to detect the presence of a nucleic acid sequence-of-interest in a sample by using a labeled polynucleotide which forms a complex with RecA and a target dsDNA without subjecting the polynucleotide sequence of the sample to denaturing conditions. In addition, as is mentioned hereinabove, since RecA-mediated exchange is fast (e.g., occurs within minutes), the use a labeled polynucleotide which forms a complex with RecA can replace known hybridization-based techniques which are intended to identify presence of nucleic acid sequences-of-interest in test samples including double strand polynucleotides (e.g., double strand genomic DNA).

Thus, according to another aspect of the present invention the method of detecting the presence of a nucleic acid sequence-of-interest is effected by: (a) providing a complex of a labeled polynucleotide which comprises a nucleic acid sequence complementary to the nucleic acid sequence-of-interest and a recombinase and; (b) incubating said complex with the sample under conditions suitable for exchange between said labeled polynucleotide and the polynucleotide sequence of the sample; and (c) measuring a rate and/or amount of said exchange to thereby detect the nucleic acid sequence-of-interest in the sample.

According to the method of this aspect of the present invention the recombinase forms a complex with the labeled polynucleotide of the present invention which comprises a nucleic acid sequence complementary to the nucleic acid sequence-of-interest. Such a complex can be formed, for example, by designing a labeled polynucleotide duplex in which a portion of one of the strands is single stranded (i.e., an overhang strand), which can form a complex (also referred to as “polymerizes” hereinafter) with the recombinase (e.g., RecA). It will be appreciated that the length of the overhang sequence is selected capable of forming a complex with the recombinase. Such a length can be for example 20-50 nucleic acids.

Following formation of a complex between the labeled polynucleotide and the recombinase, the complex is incubated with the sample under conditions suitable for exchange between the labeled polynucleotide and the polynucleotide sequence of the sample. Thus, in this configuration the invader strand is the single strand polynucleotide of the labeled polynucleotide which formed a complex with RecA and the target duplex is the polynucleotide sequence of the sample. The conditions suitable for exchange according to this aspect of the present invention are essentially as described hereinabove (e.g., hydrolysable ATP). Preferably, in this configuration such conditions are non-denaturing since no denaturation of the polynucleotide sequence of the sample is needed since the exchange can occur between a double stranded labeled probe and a double stranded unlabeled target duplex (the polynucleotide sequence of the sample). While denaturing conditions include a denaturing temperature (e.g., 95° C.) and/or a denaturing agent such as a denaturing chemical [sodium hydroxide or formaldehyde], non-denaturing conditions are those devoid of such denaturing temperature or agents and include, for example, a salt concentration which favors the formation of a double stranded duplex (e.g., physiological conditions with 150 mM sodium chloride and a physiological temperature (e.g., 35° C.-40° C.).

Preferably, the polynucleotide of the sample used by the method according to this aspect the present invention is a naturally occurring genomic DNA (e.g., a fragment of a genomic DNA) which can be either in a single strand configuration (e.g., following denaturation) or in a natural configuration as a double strand DNA (i.e., a non-denatured DNA duplex).

Alternatively, the polynucleotide of the sample used by the method according to this aspect the present invention can be a cellular RNA which either forms a secondary structure or is present as a single stranded polynucleotide.

Thus, the teachings of the method of this aspect of the present invention can be used to detect a nucleic acid sequence-of-interest in polynucleotide sequence of a sample without denaturing the double stranded polynucleotide sequences of the sample, thus avoiding the use of denaturing agents such as sodium hydroxide and/or formaldehyde which may interfere with other diagnostic tests.

It will be appreciated that when such a configuration is used (i.e., when the labeled polynucleotide forms a complex with a recombinase and a target duplex is the polynucleotide sequence of the sample), the rate and/or amount of exchange between the polynucleotide sequence of the sample and the labeled polynucleotide of the present invention can be measured in situ, i.e., within the cell or tissue where the polynucleotide sequence of the sample naturally exists (e.g., a tissue specimen such as a cell sample, a paraffin-embedded section, a cryosection and the like). According to a presently preferred embodiment of the present invention, detection of the nucleic acid sequence-of-interest is effected in situ. Thus, the method of the present invention can replace currently practiced in situ techniques such as fluorescent in situ hybridization (FISH) for identification of chromosomal abnormalities:

It will be appreciated that detection of a nucleic acid sequence-of-interest using the teachings of the present invention can be used to diagnose a variety of pathologies and/or conditions. For example, identification of disease-associated nucleic acid such as a nucleic acid sequence of a virus can be used to diagnose viral infection such as HIV, Hepatitis virus (A, B and/or C)]. For this application, the labeled polynucleotide preferably includes a nucleic acid sequence which is complementary to that of the virus causing the infection.

Alternatively, the nucleic acid sequence-of-interest may be associated with a genetic disease such as mutated nucleic acid sequence (e.g., a SNP associated with a disease, abnormal splicing, inversion, duplication, rearrangement). For example, if the mutated nucleic acid sequence is an abnormal splice variant, the labeled polynucleotide of the present invention preferably includes a nucleic acid sequence which is complementary to the disease-associated abnormal splice variant and the RecA-mediated exchange reaction will be employed on RNA molecules of the sample. For example, the splice mutation T→C in intron 20 (IVS20+6) of the genomic sequence encoding the kinase complex-associated protein (IKBKAP) (GenBank Accession No. NM 003640) which is found in a subset of patients with Familial Dysautonomia results in exon skipping and an abnormal splice variant as set forth in SEQ ID NO:44.

Still alternatively, as described in Example 6 of the Examples section which follows, the nucleic acid sequence-of-interest may be derived from a chromosomal region (e.g., a centromeric region) which can used to identify the occurrences and/or structure (e.g., presence or absence of rearrangement) of a certain chromosome and thus the method of this aspect of the present invention can be used for diagnosing chromosomal abnormalities such as abnormal chromosomal numeration (e.g., Down syndrome associated with trisomy 21), rearrangement, duplication, deletion which are associated with various pathologies (e.g., cancer) and/or genetic disorders or syndromes (e.g., DiGeorge syndrome). It will be appreciated that the detection of the nucleic acid sequence-of-interest can be also performed on fetal cells and thus the method according to this aspect of the present invention can be used to detect abnormal fetuses having a genetic disorder or syndrome by offering prenatal diagnosis.

Still alternatively, as described in Example 5 of the Examples section which follows, the nucleic acid sequence-of-interest may include a repeated nucleic acid sequence. As used herein the phrase “repeated nucleic acid sequence” refers to a nucleic acid sequence consisting of 3 or more nucleotides which appears in tandem in a nucleic acid sequence (such as in a genome of an individual) at least 3 time, at least 5 times, at least 10 times, at least 50 times, at least 100 times, at least 500 times. Non-limiting examples of such a repeated nucleic acid sequence is a CTG trinucleotide or a CAG trinucleotide sequence which is repeated to various extents in different individuals and which variations in the length of such a repeat (usually extension of the repeat) are associated with disease formation, such as with spinocerebellar ataxia type 8 [Moseley M L et al., Nat. Genet. 2006 July, 38(7):758-69. Epub 2006 Jun. 25] or Myotonic Dystrophy and Huntington's Disease [Falk M et al., Genet Test. 2006 Summer; 10(2):85-97]. Additionally or alternatively, such a repeated sequence can be the repeated nucleic acid sequence present in the telomeres of the chromosomes (e.g., as set forth in SEQ ID NO:43) and thus the method of this aspect of the present invention can be used to diagnose pathologies associated with instability of the telomeric ends such as cancer, heart disease, stroke, or infection, Dyskeratosis congenita and long-term chronic stress or infections (Harley C B, Curr Mol. Med. 2005, 5: 205-11). Similarly, as described in Example 5 of the Examples section which follows, presence of certain repeats of the telomere sequences (e.g., long telomere end) is indicative of presence of fetal cells and thus can be used for the detection of fetal cells and/or nucleic acid sequences in a cell sample (e.g., a sample derived from maternal cervix, amniocentesis or maternal blood which may include mixed populations of cells, i.e., maternal and fetal cells). It will be appreciated that for detection of a short nucleic acid repeat unit such as a repeat unit composed of 3, 4, 5, or 6 nucleotides, the labeled polynucleotide of the present invention comprises a sequence of at least 20-25 nucleotides which is complementary to several copies (usually in tandem) of the repeated unit [e.g., 8 copies of a trinucleotide repeat unit (e.g., CTG) or 4 copies of the 6-nucleotide repeat unit as set forth by SEQ ID NO:43)].

Still alternatively, as described in Example 6 of the Examples section which follows, the nucleic acid sequence-of-interest may encode for a polypeptide of the major a histocompatibility complex family (e.g., HLA-A) and thus the method of this aspect of the present invention can be used for determining tissue compatibility of donor cells to recipient cells prior to tissue or cell transplantation.

The above described agents [e.g., the recombinase, the hydrolysable ATP, the labeled polynucleotide] for detecting the presence of a nucleic acid sequence-of-interest may be included in a kit/article of manufacture preferably along with appropriate instructions for use and labels indicating FDA approval for use in vitro and/or in vivo.

Such a kit can include, for example, at least one container including at least one of the above described diagnostic agents (e.g., the labeled polynucleotide) and the general reagents (i.e., the recombinase, hydrolysable ATP and buffers) packed in another container. The kit may also include appropriate buffers and preservatives for improving the shelf-life of the kit.

It will be appreciated that such a kit can be designed for the detection of multiple nucleic acid sequences-of-interest (e.g., multiple SNPs) in one or more reactions. For example, the kit can include multiple labeled polynucleotides (or a plurality of labeled polynucleotides), each comprises a nucleic acid sequence which is complementary to the nucleic acid sequence-of-interest. As is shown in FIGS. 2a-c and is described in Example 1 of the Examples section which follows, the rate of exchange depends on the number of mismatches between the invading strand and the target duplex, e.g., the rate of exchange of two or more mismatches is slower than the rate of exchange in the presence of only one mismatch.

Accordingly, the kit can be used with multiple labeled polynucleotide sequences (which can be chemically synthesized as described hereinabove) and/or multiple polynucleotide sequences of a sample (which can be derived from RT-PCR or PCR reactions). Preferably, the multiple polynucleotide sequences of the sample are obtained from multiplex PCR reactions. Methods of preparing multiplex PCR reactions are well known in the art (Wang S H, et al., 2004, Biosens. Bioelectron. 20: 807-13; Pemov A et al., 2005, Nucleic Acids Res. 33: ell). Briefly, a genomic DNA is subject to a PCR reaction with multiple pairs of primers which are designed to anneal at a specific annealing temperature (e.g., 60° C.) to thereby achieve specific PCR products, each from one pair of primers. The PCR products are then preferably denatured (using e.g., a 5-minute incubation at 95° C., followed by a fast cooling to 4° C.) and are then mixed with the recombinase of the present invention to form multiple nucleoprotein complexes. Such complexes are then added to the labeled polynucleotides and the rate and/or amount of exchange can be measured. Alternatively, the invading strands (e.g., the labeled polynucleotide or the polynucleotide sequence of the sample), the recombinase and the target duplexes (e.g., the labeled polynucleotide or the polynucleotide sequence of the sample) are directly mixed and the rate and/or amount of exchange is measured.

It will be appreciated that in case multiple labeled polynucleotides are utilized, the rate and/or amount of exchange of each target duplex should be measured in a manner enabling individual recording. To enable individual recording of the rate and/or amount of exchange of each labeled polynucleotide, the labeled polynucleotides are preferably configured in an addressable location manner. Such addressable location manner can be achieved using various approaches.

For example, the labeled polynucleotides of the present invention can be in a liquid phase (e.g., in a vial, tube or well) with a pre-determined order. Preferably, a multi-well plate is used (e.g., 96-well plate or 384 well-plate) and the signal (resulting of FRET as described hereinabove) is measured by using a plate reader [e.g., the CytoFluor Series 4000 fluorescence multiwell plate reader (PerSeptive Biosystems, Framingham, Mass.) or the Dynex Fluorite 1000 fluorescence multiwell plate reader (Dynex Technologies, Inc., Chantilly, Va.)]. Alternatively, the labeled polynucleotides can be placed in a microfluidic system such as those described elsewhere (Thorsen T., et al., 2002, Science 298:580-584; US Patent Application Publication Nos. 2005/0118068 to Kahl J. V., 2005/0095602 to West, J. et al, 2003/0087309 to Chen, S., and 2002/0164824 to Xiao J. et al, which are fully incorporated herein by reference). Such microfluidic multiplexors are combinatorial arrays of binary valve patterns which allow complex fluid manipulations. The main advantage of using microfluid systems is the scaling down of reaction components (e.g., the ATP, recombinase and the labeled polynucleotide of the present invention), yet with a significant signal to background ratio.

Additionally or alternatively, the labeled polynucleotides of the present invention can be attached to a solid support and preferably configured in a form of an array. The phrase “solid support” refers to any substrate to which the labeled polynucleotide of the present invention can be coupled, provided that it retains its recombination (i.e., strand exchange) characteristics. Usually the solid substrate is a microsphere (bead), a magnetic bead, a nitrocellulose membrane, a nylon membrane, a glass slide, a fused silica (quartz) slide, a gold film, a polypyrrole film, an optical fiber and/or a microplate well. Such solid substrates can be used to form DNA chips using methods known in the arts (see for example, U.S. Pat. Nos. 5,445,934, 5,744,305, 5,700,637, 5,807,522 and WO Publication No. WO 98/18961). Briefly, a polynucleotide (e.g., an oligonucleotide) is synthesized on or spotted and then immobilized to a predefined region of a chip solid phase (substrate). For example, a DNA fragment is physically spotted using a pin tip onto a solid phase substrate such as a slide glass which has been subjected to special processing such as poly-L-lysine-coating or silanization. Prior to application of the polynucleotide to the solid support, the polynucleotide is preferably modified to facilitate fixation to the solid support. Such modifications may encompass homopolymer tailing, coupling with different reactive groups such as aliphatic groups, NH₂ groups, SH groups, carboxylic groups, or coupling with biotin, digoxigenin or haptens.

It will be appreciated that the labeled polynucleotide of the present invention can be attached to the solid support using several configurations. According to one preferred embodiments of the present invention, a ssDNA (e.g., an oligonucleotide) is synthesized on or attached to the solid support (e.g., a glass) in a labeled or unlabeled configuration. If an unlabeled ssDNA is utilized, then following synthesis or attachment to the solid support, the appropriate label (e.g., TAMRA or Cy5) is preferably added. Prior to incubation with the complex formed between recombinase and the invading polynucleotide, a plurality of ssDNAs (e.g., oligonucleotides) which are complementary to the labeled ssDNAs that are attached to the solid support are added to thereby form the target duplexes on the solid support. As is mentioned before, the target duplexes can be labeled on both strands or on one strand only. In order to form target duplexes which are labeled on both strands, the added ssDNAs (which are complementary to the labeled ssDNAs attached to the solid support) are also labeled (on the opposite end with respect to the attached ssDNA). Thus, in this configuration, one labeled DNA strand is attached to the solid support, while the second labeled and complementary strand is bound to the solid support by means of hydrogen bonds only.

According to another preferred embodiment of the present invention, following the attachment or synthesis of the labeled ssDNA to the solid support, unlabeled complementary ssDNAs are added to the solid support (to form semi-labeled duplexes). It will be appreciated that when this configuration is used, the invading polynucleotides which form a complex with the recombinase are preferably conjugated to a suitable FRET acceptor. As is mentioned before, such an acceptor can be a fluorophore molecule (in which case the acceptor emission can be measured) or can be a non-fluorophore molecule (in which case only the quenching of the donor fluorescence can be measured).

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific AMERICAN Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

DNA oligonucleotides and RecA protein—Single-stranded DNA oligonucleotides labeled at their 5′ or 3′-ends with either Tetramethylrhodamine (TAMRA) or Cy5 fluorescent groups (Thermo Bioscience GmbH, Ulm, Germany), were HPLC and PAGE purified. Unlabeled oligonucleotides were synthesized and purified (PAGE) by the Synthesis Unit of the Biological Services Department of the Weizmann Institute of Science. The TAMRA and Cy5 labeled complementary strands were mixed in a 1:1 ratio in 10 mM Hepes-KOH (pH 7.5), 100 mM NaCl, 10 mM MgCl₂, and were hybridized by slowly cooling from 90° C. to 20° C. over five hours. The DNA sequences used in the present study are shown in detail in FIGS. 2-f, and 5 and comprise modifications of the binding site of the integration host factor (IHF) of E. coli (Sagi et al., 2004). The labeled oligomeric sequences were designed with staggered ends at the side of the duplex bearing the donor-acceptor pair. This allowed to achieve near maximum transfer efficiency by avoiding short-distance effects in resonant energy transfer (Schuler et al., 2005). In practice, the displaced strand of the duplex was 5 bases longer from each side than the duplex strand that switches partners (see FIGS. 1a-c). Furthermore, the presence of 5 mismatches between the complementary strands at the end of the shorter partner bearing the fluorophore prevented the formation of Watson-Crick pairs there. In addition, the labeled complementary strands were homologous for 50 bp. Given that the persistence length of ssDNA is about a base pair long, donor and acceptor were effectively dangling, enhancing their rotation in space and thus averaging over their relative orientations. The sequence of the invading strands were of the same length as their complementary partner in the duplex, and could form Watson-Crick pairing at both ends, in contrast with the strands in the duplex prior strand exchange. Therefore, Watson-Crick pairing could extend for 55 by in a situation of full homology. This design introduced a small free energy bias in favor of recombination, and enabled the attainment of a fraction of strand exchange of about 80%. All ssDNA oligonucleotides used in the present study were checked for their secondary structure (Zuker, M., 2003, Nucleic Acids Research 31, 3406-3415). For the 55-mers, the requirement was to have less than eight successive intra-strand hydrogen bonds separated by fewer than four unpaired bases as by other workers (Folta-Stogniew et al., 2004). RecA protein was purchased from New England Biolabs.

Sample preparation—All FRET measurements were carried out in a reaction buffer containing 20 mM Hepes-KOH (pH 7.5), 2 mM ATP and 20 mM MgCl₂. Addition of 2 mM DTT and 100 μg/ml BSA had no measurable effect on the results. In the main protocol used in the assays of the present study, RecA, ssDNA and labeled dsDNA were mixed in a reaction buffer to final concentrations of 0-3 μM (typically 1 μM), 200 nM and 100 nM respectively, incubated for 5-10 minutes (typically 5 minutes) at 37° C., and then injected into the measuring chamber of the setup. In assays probing recombination in the presence of competitor dsDNA of partial homology, the above protocol was modified by the addition of competitor duplexes together with the other reagents. However, in order to separate the timescale of filament formation by RecA polymerization from other timescales such as those due to diffusional search and synapse lifetime, a second protocol in which filaments were preformed was used. In this protocol, ssDNA and RecA were incubated in reaction buffer at 37° C. for 5 minutes, and then the labeled dsDNA and competitor were added and incubated for another 5 minutes. Such recombination reactions were performed in the presence of 100 nM labeled DNA, 200 nM ssDNA, 2.5 μM RecA (18 monomers per 55 mer DNA), and 1 μM competitor. Both protocols yielded the same long time, steady state levels of strand exchange. It will be appreciated that recombination reactions (strand exchange) can take place with smaller concentrations of DNA and RecA and minute absolute quantities of all reagents.

FRET measurements and analysis—Both ensemble and sp-FRET measurements were performed using confocal fluorescence detection, and laser light illumination (Argon 514 nm line). The laser beam was collimated, reflected with a dichroic mirror (Omega Optical, DRLP540), and focused an objective (Zeiss 100×, NA 1.4, oil immersion), filling its back aperture. The sample is placed inside a glass chamber, with a cover-glass window facing the objective. To detect freely diffusing molecules and reduce background fluorescence, the focal point is placed about 10 μm from the glass surface. Fluorescence is collected with the same objective, and focused on a 100 μm pinhole, after residual laser light is filtered with a 514 nm optical notch-filter (HNPF-514.5-1.0, Kaiser Optical Systems). Following the pinhole, the light is divided with a second dichroic mirror (Omega Optical, DRLP630), and focused on two avalanche photodiodes (Perkin Elmer, SPCM AQR-14). Optical band-pass filters were used to increase signal-to-background ratio: (Omega Optical, 580DF30) in the donor channel, and (Omega Optical, 670DF40) in the acceptor channel. Detection is performed with a counter based on a PC counting board (National Instruments, DAQ 6602). For ensemble measurements a concentration of 90 nM DNA is used. To avoid photobleaching the sample was illuminated with 14 μW laser power. The intensities from both donor and acceptor channels were used to calculate transfer efficiency (TE) as described previously (Sagi et al., 2004). TE is the ratio of the acceptor over the sum of donor and acceptor channels, after subtracting from the acceptor channel the laser's direct excitation and the overlap between donor and acceptor. In these experiments, a decrease of donor fluorescence of 100 counts results in an increase in acceptor yield of 5-6 counts. Therefore, the factor γ, which measures the quantum yield, was estimated to be 0.05. Due to the configuration of the donor and acceptor in the labeled duplex, the donor's emission increases up to nine fold in a strand displacement assay.

The fraction of exchange was extracted from donor and acceptor intensities as follows: first, the intensities from donor (d 1) and acceptor (al) channels were measured in a sample containing prehybridized labeled duplexes. Next, the intensities from donor (d2) and acceptor (a2) channels were measured in a sample containing unhybridized, fully separated donor-labeled and acceptor-labeled ssDNA oligonucleotides, at the same concentration as in the duplex measurements. To ensure their separation during this measurement, the labeled ssDNAs were first hybridized with complementary unlabeled ssDNA partner strands. For a given RecA concentration, the assumption was that labeled ssDNA species are partitioned into doubly-labeled duplexes, or singly-labeled ones resulting from strand exchange. The fraction of doubly-labeled dsDNA undergoing strand exchange X is then calculated from the equations:

d=X·d2+(1−X)·d1

a=X·a2+(1−X)·a1

where “d” and “a” are the measured intensities of the donor and acceptor respectively.

### Example 1

### RecA-Mediated Strand Exchange is Sensitive to Single Mismatches

Experimental Results

Recombination between ssDNA and dsDNA—To assess the efficiency of strand exchange as a function of the degree of heterology between dsDNA and ssDNA-RecA nucleoprotein complexes, dsDNA oligomers, doubly-labeled at one end with donor (e.g., on the 5′-end) and acceptor (e.g., on the 3′-end) were combined with ssDNA at different RecA concentrations. FIG. 2a depicts the transfer efficiency (TE) as a function of RecA concentration, for different amounts and location of mismatches as shown by SEQ ID NOs:1, 2, 3, 4 and 5. In these experiments, the A-T content and location was preserved, while mismatches were introduced by exchanging C to G. In experiments with fully homologous ssDNA-duplexes, TE was decreased from a high value, 0.9, due to the close average proximity of donor and acceptor, to a value slightly above 0.4 at 2.5 μM RecA. This corresponds to a fraction of ˜0.6 duplexes having undergone strand exchange as shown in FIG. 2b. One or two well-separated mismatches far from the invading 3′ end of the ssDNA-RecA nucleoprotein complex did not change appreciably the efficiency of strand exchange for RecA concentrations below ˜1.5 μM, though at 2.5 μM a measurable difference in the fraction of exchange lies well above experimental error. Three well-separated mismatches led however to large changes in TE and fraction of strand exchange over all the range of RecA, while four mismatches barely caused a reduction in TE and consequently, the fraction of exchange was very small (<0.02). Eight mismatches led to an undetectable reduction of TE (data not shown). In FIG. 2c the effects of varying mismatch locations was studied in the case of one, two and three mismatches. In contrast to a single mismatch lying away from both ends (FIG. 2a), single mismatches sufficiently near the 3′ end of the invading strand (i.e., the ssDNA which form the nucleoprotein filament with RecA) resulted in a significant change in the fraction of exchange. As a control, a single mismatch near the 5′ end was shown not to affect strand exchange (FIG. 2d). Furthermore, there are significant differences in the efficiency of strand exchange when two or three mismatches are either well separated or clustered. In particular, three mismatches with two of them close by resulted in a ˜10-fold reduction in strand exchange relative to full homology. Thus a sequence divergence of 3-4 mismatches appears to provide a high enough barrier against recombination in the range of RecA concentrations of these experiments.

Significant differences in the fraction of strand exchange using a single mismatch positioned close to the staggered end of the labeled dsDNA—FIGS. 5a-b illustrate the effect of a single mismatch in the invading ssDNA located at the 5′-end of the invader strand (which is near the staggered end of the labeled dsDNA). A dsDNA containing a staggered end (nucleotide coordinates 49-55 as set forth by SEQ ID NO:19 and nucleotides 1-12 as set forth by SEQ ID NO:20) and a complementary region (nucleotide coordinates 1-48 as set forth by SEQ ID NO:19 and nucleotide coordinates 13-60 as set forth by SEQ ID NO:20) was labeled with a donor (TAMRA) and an acceptor (Cy5). To determine the effect of a single mismatch on the rate of recombination, two unlabeled invading ssDNA were used: SEQ ID NO:6, which is 100% complementary to the nucleic acid sequence set forth by SEQ ID NO:19 and is identical to nucleotide coordinates 6-60 as set forth by SEQ ID NO:20; and SEQ ID NO:18, which exhibits one mismatch as compared with SEQ ID NO:6 (a G at position 5 instead of a C; see FIG. 5a). As is shown in FIG. 5b, while in the presence of 0.75 μM RecA more than 70% of the target duplex molecules were exchanged using the oligonucleotide with the identical sequence to the displaced strand (SEQ ID NO:6), only 30% of the target duplex were exchanged using the mismatched oligonucleotide (SEQ ID NO:18). These results suggest the use of RecA-mediated strand exchange for detection of single mismatches, as in the case of SNP detection.

Analysis and Discussion

The results presented above demonstrate that RecA alone is able to detect DNA sequence divergence in recombination processes at the level of single mismatches, a much higher level of discrimination than hitherto believed (Bazemore et al., 1997). This high level of discrimination may play an important role in maintaining a minimal barrier against gratuitous recombination (Matic, I., et al., 1996, Trends in Microbiology 4, 69-73) in cases where mismatch repair systems are mutated as in subpopulations of natural E. coli isolates (Matic et al., 1995), inactive as in situations of stress (Bregeon et al., 1999), or saturated as in interspecies transformation of S. pneumoniae strains (Clayerys, J. P., et al., 2000, Molecular Microbiology 35, 251-259). Discrimination imposes a high barrier against recombination: a sequence divergence of four mismatches, suitably placed within a homology region of 50 by was enough to reduce severely the fraction of exchanges, particularly in ssDNA-duplex experiments (FIGS. 2a-c).

These results also show that the extent of RecA-catalyzed strand exchange is sensitive not only to the extent of sequence divergence, but also to the location of mismatches both in ssDNA-duplex and duplex-duplex exchanges. For instance, there is a considerable difference in the efficiency of strand exchange between well-separated pairs or triplets of mismatches, or whether they are clustered in the ssDNA-duplex experiments. In particular, these results demonstrated a higher sensitivity to mismatches when the latter are sufficiently near the 3′ end, the invading strand end (Friedman-Ohana and Cohen, 1998), than when they are farther down.

In recombination between ssDNA and duplex DNA, discrimination is the highest for RecA concentrations in the range 0.0-0.5 μM, within which RecA polymerization-depolymerization cycles on the ssDNA substrate occur (Bar-Ziv, R., et al., 2002, Proceedings of the National Academy of Sciences of the United States of America 99, 11589-11592; Bork, J. M., et al., 2001, Journal of Biological Chemistry 276, 45740-4574).

Consistent with this idea, it has been suggested that homology recognition occurs through a minimal efficient processing (MEP) segment. The increase in tₑ upon removal of RecA homologous DNAs remain paired provided the length of homology is above 26 by (Hsieh, P., et al., 1992, Proceedings of the National Academy of Sciences of the United States of America 89: 6492-6496).

### Example 2

### The Effects of Energy Dissipation on Strand Exchange

To elucidate the role energy dissipation plays in the detection of DNA sequence divergences, RecA-mediated recombination reactions were carried out in the presence of ATPγS.

Experimental Results

ATP hydrolysis is required for efficient RecA-mediated strand exchange —FIG. 2e depicts the fraction of strand exchange as a function of RecA concentration in ssDNA-dsDNA recombination experiments using a fully homologous sequence, and a sequence including two mismatches near the 3′ end in three situations: in the presence or absence of ATP and in the presence of ATPγS. ATP hydrolysis leads to efficient strand exchange which exceeds ˜60% for ˜2 μM RecA and above, in full homology experiments. In the absence of ATP there is measurable strand exchange, albeit the fraction of exchange lies below 20%, for RecA concentrations below ˜2.5 μM. Thus, energy dissipation is not strictly needed for effecting strand exchange, in agreement with previous findings (Kowalczykowski, S. C., and Krupp, R. A., 1995, Proceedings of the National Academy of Sciences of the United States of America 92, 3478-3482), although efficient strand exchange is observed only in the presence of ATP.

Experiments with two mismatches and ATP hydrolysis lead to a comparable fraction of exchange as in experiments with a fully homologous sequence, above RecA concentrations ˜2.5 and lower below this concentration. However, in the absence of energy dissipation, the degree of exchange is rather small, below 5% even for high RecA concentrations (FIG. 2e).

Experiments using the non-hydrolyzable substrate ATPγS, reported in FIG. 2e, show that the extent of strand exchange is small, for the whole range of RecA concentrations tested. An interesting and unique feature of experiments carried out with ATPγS is the large extent of the fluctuations in the measured signals, either of the donor or the acceptor fluorescence. To illustrate this, the temporal traces of donor fluorescence in the presence of ATP or ATPγS were compared (FIG. 2f). Each time point corresponds to a temporal average of the measured fluorescence over 1 second. Strikingly, the fluctuations of the fluorescence signal are much larger in the presence of ATPγS, and their typical timescale is of the order of 1 second. This behavior, characteristic of large, labeled objects crossing the focal region of the excitation beam, suggests that in the presence of both RecA and ATPγS, large aggregates of labeled DNA are formed.

Discussion

Without ATP hydrolysis, RecA is unable to recycle between a state with high binding affinity to ssDNA, and a state with low binding affinity favoring depolymerization from the ssDNA substrate (Rosselli, W., and Stasiak, A., 1990, Journal of Molecular Biology 216, 335-352). Furthermore, it is known that ATPγS increases the affinity of RecA for ssDNA, and that a RecA monomer has two binding sites for DNA (Kubista, M., et al., 1990, Journal of Biological Chemistry 265, 18891-18897; Mazin, A. V., and Kowalczykowski, S. C., 1998, Embo Journal 17, 1161-1168; Muller, B., et al., 1990, Journal of Molecular Biology 212, 97-112; Zaitsev E. N. and Kowalczykowski, S. C. 1999, Journal of Molecular Biology 287, 21-31). Hence, in the presence of ATPγS, RecA-ssDNA filaments are stable and act as molecular stickers, linking together a number of labeled dsDNA molecules and forming a gel (Tsang, S. S., et al., 1985, Biochemistry 24, 3226-3232; Dutreix, M., et al., 2003 ComPlexUs 1, 89-99).

### Example 3

### The Effect of Partially Homologous, Competitor Sequences on RecA-Mediated Recombination

The higher sensitivity of strand exchange to mismatches near the invading strand end of the nucleoprotein complex, as revealed by the results described in Examples 1 and 2 hereinabove, suggests that the stability of recombination intermediates formed after invasion of the 3′ end plays an important role during the search for homology. In addition, in vivo recombination measurements of phage-plasmid cointegrates in E. coli have revealed that for recombination to be efficient, the length over which homology extend must lie above a minimal value, the minimal efficient processing segment (MEPS) (Shen, P., and Huang, H. V., 1986, Genetics 112, 441-457). It was hypothesized that physical constraints on the substrate length for recombination, determined most likely by RecA requirements, may account for the length of the MEPS.

Experimental Results

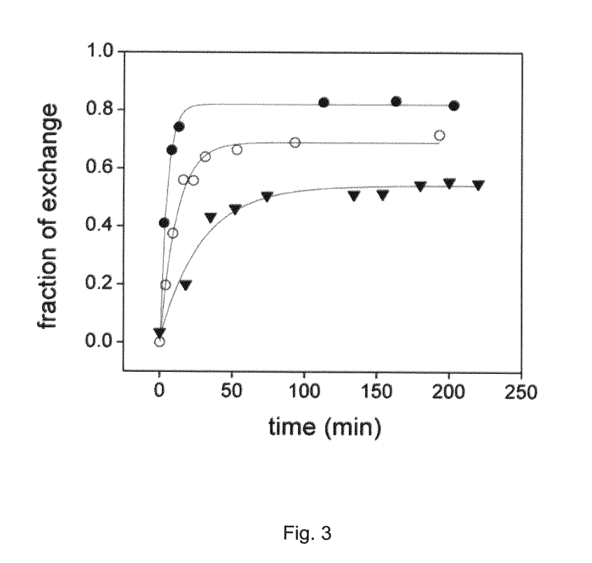

Homology search in the presence of competing, partially-homologous sequences—To shed light on the process of homology search and its characteristic timescales, test for the stability of recombination intermediates, and find out about RecA-related molecular mechanisms behind the minimal efficient processing segment (MEPS), experiments in which RecA-ssDNA nucleoprotein complexes search for their fully homologous partners within labeled duplexes were conducted in the presence of competing, unlabeled dsDNA oligomers of the same length (55 bp), but bearing homology only to tracts of different lengths including the 3′ end of the nucleoprotein complexes (see FIG. 1c). In these assays, the concentration of the competitor duplexes was tenfold that of the fully homologous labeled duplexes, while the RecA concentration was enough to polymerize on only half the ssDNA oligomers. FIG. 3 illustrates the fraction of labeled duplexes undergoing strand exchange (f) as a function of time, under competition with different duplexes whose length of homology ranged from 0 to 35 bp. Typically, the behavior is characterized by a fast rise at early times, and levels off at long times. Both the rise time and the saturation value depend on the length of homology of the competitor DNA: when the length of homology is large, a competitor is more successful at slowing down the rate at which the ssDNA finds its fully homologous partner, and furthermore, a larger fraction of the ssDNA is sequestered by the competitor at long times. These results argue that an exponential dependence provides a good approximation for the observed increase in f.

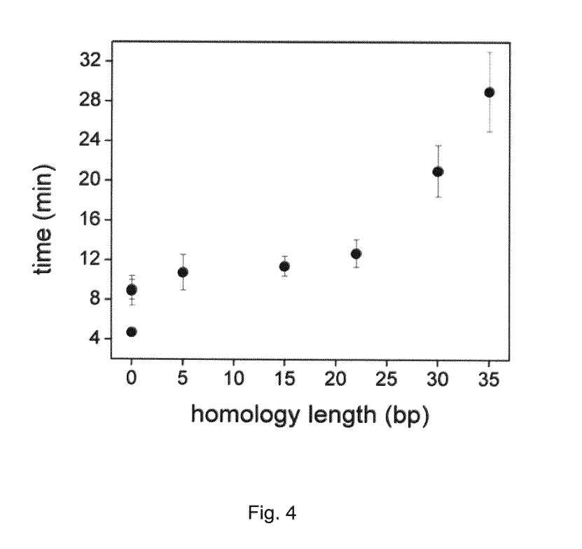

Accordingly, the curves in FIG. 3 were fit with an exponential dependence in order to extract tₑ as a measure of the rise time for the different competitors. The fits describe well the data, including the long-time behavior. FIG. 4b depicts tₑ as a function of the length of homology x of the competitor duplexes. For comparison purposes, the value of tₑ in the absence of competitor is also plotted (FIG. 4). There are two salient features in the behavior of tₑ: first, a very weak increase with x for small values of this quantity, followed by a higher rate of increase when x>25 bp. Note that even for x=0, i.e., no homology with the competitor duplexes, strand exchange is delayed by a significant amount (−4 minutes). Given the experimental procedure followed in these experiments, tₑ subsumes contributions due to the timescale associated with ssDNA-RecA filament formation by RecA nucleation and polymerization, the diffusional search of the filament for its duplex partner, synapse lifetime, and finally the time it takes for strand exchange. To factor out filament formation, further experiments were conducted in which filaments were formed prior to adding the labeled duplex. In the case of no competitor homology, these experiments yield tₑ=1.9±0.3 minutes, instead of the 4.7±0.3 minutes measured when following the first procedure. The difference in tₑ between an experiment in which no competitor is present, to one in which competitor bearing no homology is present allows the separation of the synapse lifetime from the process of strand exchange and estimate the former. This estimate is an important ingredient to understand the process of homology search within a cell. Thus, these results show that the timescale characterizing diffusional search between the filament and the labeled duplexes is negligible.

These results suggest that RecA-mediated strand exchange can be used on multiplex-PCR reaction selected such that the level of homology between the multiplex PCR products does not exceed 20-25 bp.

### Example 4

### Testing for SNP in a Nucleic Acid Sequence without Sample Denaturation

The biologically active form of DNA is present as a double-stranded DNA in a Watson-Crick structure. Such a duplex structure is maintained even following DNA extraction from cells. In addition, the simplest way to amplify a specific sequence out of a genome is by the use of PCR, which also results in a duplex DNA product. On the other hand, all sequence analyzing methods in which a specific nucleic acid sequence and/or sequence alteration (substitution) are identified involve DNA denaturation in order to enable probe annealing (hybridization) to the target DNA template. For denaturation the DNA is either subjected to high temperatures (e.g., 95° C.) or is subject to alkaline conditions (e.g., NaOH). While subjecting the DNA to high temperature is not always possible when the DNA is part of a cellular or tissue structure, the use of alkaline conditions may complicate further analyses. Thus, it is therefore desirable to probe genomic DNA for the presence or absence of a given sequence or sequence alteration using DNA in a duplex form, without the need of denaturation, paving the way for PCR-free testing.

Towards this goal the present inventors have devised doubly-labeled DNA-FRET probes with a single stranded overhang, whose RecA-induced homologous recombination with a given unlabelled genomic tract results in the loss of FRET, as follows.

Materials and Experimental Methods

Design of labeled probes The labeled probe (invader double-stranded DNA) is schematically illustrated in FIG. 7b and is composed of a 69-mer oligonucleotide (SEQ ID NO:29) labeled at the 5′-end with Cy5 and a 31-mer oligonucleotide (SEQ ID NO:30) labeled at the 3′-end with TAMRA. The 31-mer overhang of the oligonucleotide set forth by SEQ ID NO:29 interacts with RecA prior to the addition of the unlabeled target duplex.

Design of unlabeled target duplexes—The two unlabeled target duplexes are double-stranded polynucleotides, each representing a perfect duplex (no mismatches between the two strands forming the double strand DNA). The two polynucleotides can be identical to each other (i.e., with no SNP with respect to each other) or can vary from each other in at least one nucleotide (i.e., the SNP nucleotide). These target duplexes represent two genomic sequences of two homologous chromosomes in a single cell (i.e., a diploid genome).

Full homology duplex—A target duplex which is homologous to the 69-mer Cy5-labeled probe (SEQ ID NO:29) is designed by the oligonucleotides set forth by SEQ ID NOs:31-32.

SNP at position 1—A target duplex which contains one SNP with respect to the 69-mer Cy5-labeled probe (SEQ ID NO:29) is designed by the oligonucleotides set forth by SEQ ID NOs:33-34. In this duplex, the polymorphic nucleotide is at position 1 with respect to the 5′-end of the TAMRA-labeled oligonucleotide set forth by SEQ ID NO:30 (see yellow-marked nucleotide in FIG. 7a).

SNP at position 6—A target duplex which contains one SNP with respect to the 69-mer Cy5-labeled probe (SEQ ID NO:29) is designed by the oligonucleotides set forth by SEQ ID NOs:35-36. In this duplex, the polymorphic nucleotide is at position 6 with respect to the 5′-end of the TAMRA-labeled oligonucleotide set forth by SEQ ID NO:30 (see green-marked nucleotide in FIG. 7a).

SNP at position 7—A target duplex which contains one SNP with respect to the 69-mer Cy5-labeled probe (SEQ ID NO:29) is designed by the oligonucleotides set forth by SEQ ID NOs:37-38. In this duplex, the polymorphic nucleotide is at position 6 with respect to the 5′-end of the TAMRA-labeled oligonucleotide set forth by SEQ ID NO:30 (see light blue-marked nucleotide in FIG. 7a).

SNP at position 8—A target duplex which contains one SNP with respect to the 69-mer Cy5-labeled probe (SEQ ID NO:29) is designed by the oligonucleotides set forth by SEQ ID NOs:39-40. In this duplex, the polymorphic nucleotide is at position 6 with respect to the 5′-end of the TAMRA-labeled oligonucleotide set forth by SEQ ID NO:30 (see grey-marked nucleotide in FIG. 7a).

SNP at position 11—A target duplex which contains one SNP with respect to the 69-mer Cy5-labeled probe (SEQ ID NO:29) is designed by the oligonucleotides set forth by SEQ ID NOs:41-42. In this duplex, the polymorphic nucleotide is at position 6 with respect to the 5′-end of the TAMRA-labeled oligonucleotide set forth by SEQ ID NO:30 (see purple-marked nucleotide in FIG. 7a).

RecA-mediated exchange—was performed essentially as described in the general materials and experimental methods of the Examples section and the fraction of exchanged polynucleotides was measured as a function of the position of the polymorphic nucleotide with respect to the 5′-end of the TAMRA-labeled oligonucleotide set forth by SEQ ID NO:30.

Experimental Results

RecA-polymerized labeled probe can induce exchange with an unlabeled target duplex such as a genomic double-stranded DNA—As is schematically shown in FIG. 7b, RecA polymerizes on the single-stranded overhang of the labeled double-stranded probe prior to the final reaction with the double-stranded DNA segment (the target duplex) representing a portion of the genome probed. The DNA-FRET probe is asymmetric, consisting of a 31 by strand labeled with TAMRA [donor (d); SEQ ID NO:30], and a longer, partially complementary strand (69 bp) labeled with Cy5 [acceptor (a); SEQ ID NO:29]. The single-stranded overhang is of 34 nucleotides, allowing polymerization of RecA protein and consequent invasion to the unlabeled duplex target. Following invasion to the unlabeled target duplex, one of the unlabeled strands hybridizes with the acceptor-labeled polynucleotide (SEQ ID NO:29) while the other strand hybridizes with the donor-labeled polynucleotide (SEQ ID NO:30), thus creating a measurable FRET signal.

RecA-mediated exchange is depended on the position of the polymorphic nucleotide in the unlabeled target duplex—FIG. 8 shows the effect of single mismatches on the fraction of exchange, as a function of their distance from the 5′-end of the 31 mer labeled oligomer (SEQ ID NO:30). It is emphasized that the unlabeled duplex DNA being tested has no mismatches between its strands. The protocol for introduction of a mismatch is as follows: One inserts a SNP at position “n” in the unlabeled 69 mer oligomer (see arrow in FIG. 7b), which is homologous to the 69 mer (acceptor; SEQ ID NO:29), labeled probe. Each unlabeled target duplex includes one SNP at a different location [e.g., in nucleotide 1, 6, 7, 8 and 11 with respect to the 5′-end of the 31 mer labeled oligomer (SEQ ID NO:30)]. The effect of the position of the SNP on the fraction of exchange is determined by comparing the fraction of exchange of each unlabeled target duplex (with a SNP; e.g., the target duplex set forth by SEQ ID NOs:41-42) to that of the full homology target duplex (SEQ ID NOs:31-32). The efficiency of recombination turns out to be strongly dependent on the presence of single mismatch differences on the double-stranded portion of the probe, relative to the duplex tested. Note that the fraction of exchange increases when the position of SNP is closer to the 5′ end of the labeled polynucleotide.

Altogether, these results demonstrate a new configuration of the RecA mediated exchange in which the labeled probe can invade into unlabeled target duplex such as the genomic DNA present in a cell. Thus, these results demonstrate the use of RecA-mediated exchange for detecting specific sequences of genomic DNA fragments, which can replace common hybridization and SNP-detection methods.

### Example 5

### Probing Telomere Length to Score for Cancer and Genetic Disorders in Fetuses

The ends of linear human chromosomes contain specialized structures called telomeres, which consist of a tandem of repeat units (TTAGGG), that help to maintain chromosomal integrity and provide a buffer of potentially expendable DNA. The length of telomeres is highly regulated and protected by the formation of a high-structure nucleoprotein complex, in which the DNA adopts a lariat-like conformation. This prevents the recognition of the chromosome end as a double-stranded break. Linear chromosomal DNA lagging-strand replication typically results in shortening of telomere length, but this latter can increase as well through the addition of further repeats by the action of an enzyme called telomerase. Telomerase gradually becomes inactive with age in most tissues (a notable exception are hematopoietic cells). Thus the overall telomere length ranges from ˜15 kbp at birth, to about 5 kbp in senescent cells. The ability to bypass replicative senescence and thus enable unlimited proliferative capacity is believed to be a rate-limiting step towards the development of malignancies including cancer and other chronic diseases such as ulcerative colitis, leading typically to too-short telomeres. Indeed, altered telomere lengths are a telltale sign of cancer. Thus the measurement of telomere length can reliably give information about whether the cell is in a healthy or diseased state.

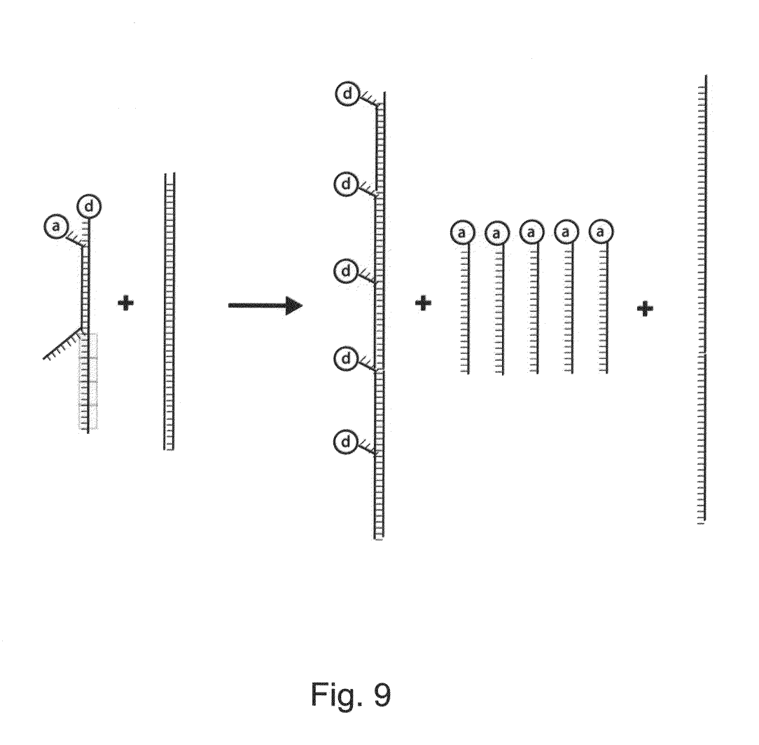

Double-stranded labeled DNA-FRET probes (such as those schematically illustrated in FIG. 7b) or single stranded fluorophore-labeled probes (e.g., fluorescein conjugated single strand oligonucleotide) can identify telomeres repeats reliably when probe design includes few (about 5) units of the basic repeat 5′-TTAGGG (SEQ ID NO:43). In this particular embodiment, variations in the number of labeled probes that attach to telomeres provide a reliable measurement of telomere length (FIG. 9).

The state of telomeric lengths can also be used to assay for the integrity of chromosomal DNA in various pathologies associated with telomere instability such as cancerous tumors (characterized with longer telomeres), heart disease, stroke, or infection (associated with shorter than average telomere length), Dyskeratosis congenita (associated with shortened telomeres due to mutations in telomerase components) and long-term chronic stress or infections (associated with accelerated telomere shortening compared to age-matched counterparts) as described in Harley C B, Curr Mol. Med. 2005, 5: 205-11.

Additionally, the state of telomeric length can be used to diagnose fetal cells and/or nucleic acid sequences (e.g., of trophoblasts shed from the maternal uterine or cervix). Since fetal cells exhibit considerably longer telomeres than maternal cells, the length of the telomere is indicative for the presence of fetal cells or nucleic acids.

### Example 6

### Uses of RecA-Mediated Exchange on Non-Denatured Duplex DNA for Detection of a Nucleic Acid Sequence of Interest

SNP detection—The ability of RecA to detect single mismatches can be used to detect SNPs in a target duplex DNA without needing to denature the polynucleotide of the sample, i.e., without subjecting the sample to denaturing conditions such as heating, incubation with sodium hydroxide or formaldehyde.

Acceleration of fluorescence in situ hybridization assays (FISH)—The ability of RecA to recognize a target sequence along a genome can be used to accelerate fluorescence in situ hybridization assays (FISH), which are routinely used to probe chromosomal disorders. FISH typically requires a few hours due to the slow pace of the uncatalyzed, hybridization step. The power of RecA induced target location and fidelity can be brought to bear on this problem. Strand exchange of chromosomal DNA with the specially-designed double-stranded labeled probes of the present invention can ascertain for example, the existence of chromosomal translocations or other genetic disorders. RecA can accelerate target location so the process can be finished in a timescale of minutes.

Tissue identification—The presence of a certain nucleic acid sequence in a sample such as of a major histocompatibility complex (MHC) can be determined using RecA-mediated exchange. As is shown in Example 4, hereinabove, a labeled probe consisting of a double strand of two oligonucleotides with an overhang capable of polymerization with RecA (i.e., formation of a complex between RecA and the labeled polynucleotide probe) can be designed such that the nucleic acid sequence of the labeled polynucleotide probe is complementary to the nucleic acid sequence-of-interest [e.g., to the nucleic acid sequence of HLA-A (GenBank Accession No. NM_{—}002116.5) or HLA-B (GenBank Accession No. NM_{—}005514.5)]. Thus, the method of the present invention enables a fast (within minutes, e.g., 10-30 minutes) detection of a specific nucleic acid sequence in a sample using simple sample preparation. The idea is to take the genomic DNA, and mix it (even without PCR) with the double-stranded labeled probe(s) of the present invention and let RecA run the exchange process. In an alternative way, the labeled probe and RecA are added in situ to a tissue section or cells without needing to subject the genomic DNA of the sample to denaturing conditions. The ability of the recombinase (e.g., RecA) protein to locate its target of a noisy environment with high signal to noise ratio may allow a very simple assay for detecting the existence of a nucleic acid sequence-of-interest. This assay is useful for example, in the case of tissue identification for compatibility of donor-recipient individuals.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Additional References are Cited in Text

US Patent Application Publication No. 2003/0180746 to Kmiec et al.
U.S. Pat. No. 6,849,410 to Shigemori et al.
Bar-Ziv, R., and Libchaber, A. (2001). Effects of DNA sequence and structure on binding of RecA to single-stranded DNA. Proceedings of the National Academy of Sciences of the United States of America 98, 9068-9073.
Bazemore, L. R., FoltaStogniew, E., Takahashi, M., and Radding, C. M. (1997). RecA tests homology at both pairing and strand exchange. Proceedings of the National Academy of Sciences of the United States of America 94, 11863-11868.
Bucka, A., and Stasiak, A. (2001). RecA-mediated strand exchange traverses substitutional heterologies more easily than deletions or insertions. Nucleic Acids Research 29, 2464-2470.
Flory, J., and Radding, C. (1982). Cell 28, 747-756.
Folta-Stogniew, E., O'Malley, S., Gupta, R., Anderson, K. S., and Radding, C. M. (2004). Exchange of DNA base pairs that coincides with recognition of homology promoted by E-coli RecA protein. Molecular Cell 15, 965-975.
Friedman-Ohana, R., and Cohen, A. (1998). Heteroduplex joint formation in Escherichia coli recombination is initiated by pairing of a 3′-ending strand. Proceedings of the National Academy of Sciences of the United States of America 95, 6909-6914.
Gumbs, O. H., and Shaner, S. L. (1998). Three mechanistic steps detected by FRET after presynaptic filament formation in homologous recombination. ATP hydrolysis required for release of oligonucleotide heteroduplex product from RecA. Biochemistry 37, 11692-11706.
Gupta, R. C., Golub, E. I., Wold, M. S., and Radding, C. M. (1998). Polarity of DNA strand exchange promoted by recombination proteins of the RecA family. Proceedings of the National Academy of Sciences of the United States of America 95, 9843-9848.
Rao, B. J., and Radding, C. M. (1994). Formation of Base Triplets by Non-Watson-Crick Bonds Mediates Homologous Recognition in Reca Recombination Filaments. Proceedings of the National Academy of Sciences of the United States of America 91, 6161-6165.
Rice et al., 2004, Genome Res. 14:116-125.
Worth, L., Clark, S., Radman, M., and Modrich, P. (1994). Mismatch Repair Proteins Muts and Mutl Inhibit Reca-Catalyzed Strand Transfer between Diverged Dnas. Proceedings of the National Academy of Sciences of the United States of America 91, 3238-3241.

1. A method of detecting a nucleic acid sequence-of-interest in a sample, the method comprising:
(a) providing a complex of a polynucleotide sequence of the sample and a recombinase;
(b) incubating said complex with a labeled polynucleotide which comprises a nucleic acid sequence complementary to the nucleic acid sequence-of-interest under conditions suitable for exchange between said polynucleotide sequence and said labeled polynucleotide; and
(c) measuring a rate and/or amount of said exchange to thereby detect the nucleic acid sequence-of-interest in the sample.

2. A kit for detecting a nucleic acid sequence-of-interest in a sample, the kit comprising packaging materials and at least one agent identified by said packaging material as being suitable for measuring a rate and/or amount of a recombinase-mediated exchange between a polynucleotide sequence of the sample and a labeled polynucleotide which comprises a nucleic acid sequence complementary to the nucleic acid sequence-of-interest.

3. The method of claim 1, wherein the nucleic acid sequence-of-interest comprises a single nucleotide polymorphism (SNP).

4. A method of detecting a nucleic acid sequence-of-interest in a sample, the method comprising:
(a) providing a complex of a labeled polynucleotide which comprises a nucleic acid sequence complementary to the nucleic acid sequence-of-interest and a recombinase and;
(b) incubating said complex with the sample under conditions suitable for exchange between said labeled polynucleotide and the polynucleotide sequence of the sample; and
(c) measuring a rate and/or amount of said exchange to thereby detect the nucleic acid sequence-of-interest in the sample.

5. A kit for detecting a nucleic acid sequence-of-interest in a sample, the kit comprising packaging materials and at least one agent identified by said packaging material as being suitable for measuring a rate and/or amount of a recombinase-mediated exchange between a labeled polynucleotide which comprises a nucleic acid sequence complementary to the nucleic acid sequence-of-interest and a polynucleotide sequence of the sample.

6. The method of claim 1, wherein said recombinase is selected from the group consisting of RecA, Rad51, DMC1, Sin, Cre, RadA and Rec12.

7. The method of claim 1, wherein the polynucleotide sequence is a PCR product.

8. The method of claim 4, wherein said polynucleotide sequence of the sample is a non-denatured fragment of a genomic nucleic acid sequence.

9. The method of claim 4, wherein said measuring of said rate and/or said amount of said exchange is effected in situ.

10. The method of claim 1, wherein said polynucleotide sequence of the sample is a single strand DNA (ssDNA).

11. The method of claim 4, wherein said polynucleotide sequence of the sample is a double strand DNA (dsDNA).

12. The method of claim 4, wherein said detecting is effected in situ.

13. The method of claim 1, wherein said incubating is effected for a time period selected from the range of 1-20 minutes.

14. The method of claim 1, wherein said conditions comprise hydrolysable ATP.

15. The method of claim 1, wherein said labeled polynucleotide which comprises said nucleic acid sequence complementary to the nucleic acid sequence-of-interest is a double stranded DNA (dsDNA) and whereas one strand of said dsDNA is labeled with a fluorescent acceptor and a second strand of said dsDNA is labeled with a fluorescent donor.

16. The method of claim 1, wherein said labeled polynucleotide which comprises said nucleic acid sequence complementary to the nucleic acid sequence-of-interest is a dsDNA and whereas one strand of said dsDNA is labeled at a 5′-end and a second strand of said dsDNA is labeled at a 3′-end.

17. The method of claim 15, wherein said fluorescent acceptor and said fluorescent donor are positioned on said dsDNA such that an average physical distance therebetween is selected from the range of 30-60 Angstrom.

18. (canceled)

19. The method of claim 1, wherein said labeled polynucleotide which comprises said nucleic acid sequence complementary to the nucleic acid sequence-of-interest is labeled on one strand and whereas said polynucleotide sequence of the sample is labeled on one strand.

20. The method of claim 1, wherein said labeled polynucleotide which comprises said nucleic acid sequence complementary to the nucleic acid sequence-of-interest is a plurality of labeled polynucleotides and whereas said plurality of labeled polynucleotides are configured as an array.

21. The method of claim 1, wherein said labeled polynucleotide which comprises said nucleic acid sequence complementary to the nucleic acid sequence-of-interest is attached to a solid support.

22. The method of claim 1, wherein the nucleic acid sequence-of-interest is selected from the group consisting of a repeated nucleic acid sequence, a disease-associated nucleic acid sequence and/or a genomic fragment of a chromosome.

23. The method of claim 3, for detecting a presence of said SNP, telomeric instability, and/or DNA or chromosomal aberrations.

24. The method of claim 4, wherein the nucleic acid sequence-of-interest comprises at least 25 nucleic acids.

25. The method of claim 4, wherein said conditions comprise non-denaturing conditions.

26. The method of claim 4, wherein said labeled polynucleotide which comprises said nucleic acid sequence complementary to the nucleic acid sequence-of-interest is a double stranded DNA (dsDNA) and whereas one strand of said dsDNA is labeled with a fluorescent acceptor and a second strand of said dsDNA is labeled with a fluorescent donor.

27. The method of claim 4, wherein said labeled polynucleotide which comprises said nucleic acid sequence complementary to the nucleic acid sequence-of-interest is a dsDNA and whereas one strand of said dsDNA is labeled at a 5′-end and a second strand of said dsDNA is labeled at a 3′-end.

28. The method of claim 4, wherein the nucleic acid sequence-of-interest is selected from the group consisting of a repeated nucleic acid sequence, a disease-associated nucleic acid sequence and/or a genomic fragment of a chromosome.

29. The method of claim 4, for detecting a presence of a SNP, telomeric instability, and/or DNA or chromosomal aberrations.


SEQUENCE LISTING (45 sequences)

SEQ ID NO: 1 - DNA, 55 bp, Artificial sequence
  Single strand DNA oligonucleotide
tagacctgttcgttgcaacaaattgataagcaatgcttttttataatgcagtgaa

SEQ ID NO: 2 - DNA, 55 bp, Artificial sequence
  Single strand DNA oligonucleotide
tagacctgttcgttccaacaaattgataagcaatgcttttttataatgcagtgaa

SEQ ID NO: 3 - DNA, 55 bp, Artificial sequence
  Single strand DNA oligonucleotide
tagacctgttcgttccaacaaattgataaggaatgcttttttataatgcagtgaa

SEQ ID NO: 4 - DNA, 55 bp, Artificial sequence
  Single strand DNA oligonucleotide
tagacctgttggttgcaacaaattcataagcaatggttttttataatgcagtgaa

SEQ ID NO: 5 - DNA, 55 bp, Artificial sequence
  Single strand DNA oligonucleotide
tagacctgttccttcgaacaaattgataaggaatgcttttttataatgcagtgaa

SEQ ID NO: 6 - DNA, 55 bp, Artificial sequence
  Single strand DNA oligonucleotide
tagacctgttcgttgcaacaaattgataagcaatgcttcttgataatgcagtgaa

SEQ ID NO: 7 - DNA, 55 bp, Artificial sequence
  Single strand DNA oligonucleotide
tagacctgttcgttgcaacaaattgataagcaatgcttcttgataatgcagtcaa

SEQ ID NO: 8 - DNA, 55 bp, Artificial sequence
  Single strand DNA oligonucleotide
tagacctgttcgttgcaacaaattgataagcaatgcttcttgataatgcactgaa

SEQ ID NO: 9 - DNA, 55 bp, Artificial sequence
  Single strand DNA oligonucleotide
tagacctgttcgttgcaacaaattgataagcaatgcttcttgataatggactgaa

SEQ ID NO: 10 - DNA, 55 bp, Artificial sequence
  Single strand DNA oligonucleotide
tagacctgttccttccaacaaattgataaggaatgcttttttataatgcagtgaa

SEQ ID NO: 11 - DNA, 55 bp, Artificial sequence
  Single strand DNA oligonucleotide
ttcactgcattatcaagaagcattgcttatcaatttgttgcaacgaacgagcaac

SEQ ID NO: 12 - DNA, 55 bp, Artificial sequence
  Single strand DNA oligonucleotide
ttcagtgcattatcaagaagcattgcttatcaatttgttgcaacgaacgagcaac

SEQ ID NO: 13 - DNA, 75 bp, Artificial sequence
  Single strand DNA oligonucleotide
atggtgcaccttgcattcactgcattatcaagaagcattgcttatcaatttgttgcaacgaacgagcaacgtcta

SEQ ID NO: 14 - DNA, 55 bp, Artificial sequence
  Single strand DNA oligonucleotide
tagacctgttcgttccaacaaattgataagcaatgcttcttgataatgcagtgaa

SEQ ID NO: 15 - DNA, 75 bp, Artificial sequence
  Single strand DNA oligonucleotide
atggtgcaccttgcattcactgcattatcaagaagcattgcttatcaatttgttggaacgaacgagcaacgtcta

SEQ ID NO: 16 - DNA, 55 bp, Artificial sequence
  Single strand DNA oligonucleotide
tagacctgttcgttccaacaaattgataaggaatgcttcttgataatgcagtgaa

SEQ ID NO: 17 - DNA, 75 bp, Artificial sequence
  Single strand DNA oligonucleotide
atggtgcaccttgcattcactgcattatcaagaagcattccttatcaatttgttggaacgaacgagcaacgtcta

SEQ ID NO: 18 - DNA, 55 bp, Artificial sequence
  Single strand DNA oligonucleotide
tagagctgttcgttgcaacaaattgataagcaatgcttcttgataatgcagtgaa

SEQ ID NO: 19 - DNA, 55 bp, Artificial sequence
  Single strand DNA oligonucleotide
ttcactgcattatcaagaagcattgcttatcaatttgttgcaacgaacaggtcta

SEQ ID NO: 20 - DNA, 65 bp, Artificial sequence
  Single strand DNA oligonucleotide
tagacgttgctcgttcgttgcaacaaattgataagcaatgcttcttgataatgcagtgaatgcaa

SEQ ID NO: 21 - DNA, 55 bp, Artificial sequence
  SNP containing DNA fragment
ttcactgcattatcaagaagcattgcttatcaatttgttgcaacgaacagntcta

SEQ ID NO: 22 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 23 - DNA, 2254 bp, Homo sapiens
gggaattctgaaagccgctggcggaccgcgcgcagcggccagagaccgagccctaaggagagtgcggcgcttcccgaggcgtgcagctgggaactgcaactcatctgggttgtgcgcagaaggctggggcaagcgagtagagaagtggagcgtaagccaggggcgttgggggccgtgcgggtcgggcgcgtgccacgcccgcggggtgaagtcggagcgcggggcctgctggagagaggagcgctgcggaccgagtaatggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatctgaggaaaggaagaggggaaaccagaatctgcaaaatctacgactctccctgtcttcctgaagctgaagctatgttcgccattaatgcagatggagtgggagatgccaaagactgaatcattgggtttttcctctgttaaaaaccttaagtgctgcagcctaatgagagtgcactgctccctggggttctctacaggcctcttcctgttgtgactgccaggataaagcttccgggaaaacagctattatatcagcttttctgatggtataaacaggagacaggtcagtagtcacaaactgatctaaaatgtttattccttctgtagtgtattaatctctgtgtgttttctttggttttggaggaggggtatgaagtatctttgacatggtgccttaggaatgacttgggtttaacaagctgtctactggacaatcttatgtttccaagagaactaaagctggagagacctgacccttctctcacttctaaattaatggtaaaataaaatgcctcagctatgtagcaaagggaatgggtctgcacagattctttttttctgtcagtaaaactctcaagcaggtttttaagttgtctgtctgaatgatcttgtgtaagggtttggttatggagtcttgtgccaaacctactaggccattagcccttcaccatctacctgcttggtctttcattgctaagactaactcaagataatcctagagtcttaaagcatttcaggccagtgtggtgtcttgcgcctgtactcccagcactttgggaggccgaggcaggtggatcgcttgagccaggagttttaagtccagcttggccaagatggtgaaatcccatctctacaaaaaatgcagaacttaatctggacacactgttacacgtgcctgtagtcccagctactctatagcctgaggtgggagaatcacttaagcctggaaggtggaagttgcagtgagtcgagattgcactgctgcattccagccagggtgacagagtgagaccatgtttcaaacaagaaacatttcagagggcaagtaaacagatttgattgtgaggcttctaataaagtagttattagtagtg

SEQ ID NO: 24 - DNA, 1391 bp, Escherichia coli
agagaagcctgtcggcaccgtctggtttgcttttgccactgcccgcggtgaaggcattacccggcgggatgcttcagcggcgaccgtgatgcggtgcgtcgtcaggctactgcgtatgcattgcagaccttgtggcaacaatttctacaaaacacttgatactgtatgagcatacagtataattgcttcaacagaacatattgactatccggtattacccggcatgacaggagtaaaaatggctatcgacgaaaacaaacagaaagcgttggcggcagcactgggccagattgagaaacaatttggtaaaggctccatcatgcgcctgggtgaagaccgttccatggatgtggaaaccatctctaccggttcgctttcactggatatcgcgcttggggcaggtggtctgccgatgggccgtatcgtcgaaatctacggaccggaatcttccggtaaaaccacgctgacgctgcaggtgatcgccgcagcgcagcgtgaaggtaaaacctgtgcgtttatcgatgctgaacacgcgctggacccaatctacgcacgtaaactgggcgtcgatatcgacaacctgctgtgctcccagccggacaccggcgagcaggcactggaaatctgtgacgccctggcgcgttctggcgcagtagacgttatcgtcgttgactccgtggcggcactgacgccgaaagcggaaatcgaaggcgaaatcggcgactctcacatgggccttgcggcacgtatgatgagccaggcgatgcgtaagctggcgggtaacctgaagcagtccaacacgctgctgatcttcatcaaccagatccgtatgaaaattggtgtgatgttcggtaacccggaaaccactaccggtggtaacgcgctgaaattctacgcctctgttcgtctcgacatccgtcgtatcggcgcggtgaaagagggcgaaaacgtggtgggtagcgaaacccgcgtgaaagtggtgaagaacaaaatcgctgcgccgtttaaacaggctgaattccagatcctctacggcgaaggtatcaacttctacggcgaactggttgacctgggcgtaaaagagaagctgatcgagaaagcaggcgcgtggtacagctacaaaggtgagaagatcggtcagggtaaagcgaatgcgactgcctggctgaaagataacccggaaaccgcgaaagagatcgagaagaaagtacgtgagttgctgctgagcaacccgaactcaacgccggatttctctgtagatgatagcgaaggcgtagcagaaactaacgaagatttttaatcgtcttgtttgatacacaagggtcgcatctgcggcccttttgcttttttaagttgtaaggatatgccatgacagaatcaacatcccgtcg

SEQ ID NO: 25 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 26 - DNA, 65 bp, Artificial sequence; identical to SEQ ID NO: 20
  Single strand DNA oligonucleotide

SEQ ID NO: 27 - DNA, 55 bp, Artificial sequence; identical to SEQ ID NO: 6
  Single strand DNA oligonucleotide

SEQ ID NO: 28 - DNA, 55 bp, Artificial sequence; identical to SEQ ID NO: 18
  Single strand DNA oligonucleotide

SEQ ID NO: 29 - DNA, 69 bp, Artificial sequence
  Single strand DNA oligonucleotide
tagacatctcctgttcgttgcaacaaattgataagcaatgcttttttataatgcatgcaagtgaaacct

SEQ ID NO: 30 - DNA, 31 bp, Artificial sequence
  Single strand DNA oligonucleotide
cgaattcaatttgttgcaacgaacaggtcta

SEQ ID NO: 31 - DNA, 69 bp, Artificial sequence; identical to SEQ ID NO: 29
  Single strand DNA oligonucleotide

SEQ ID NO: 32 - DNA, 69 bp, Artificial sequence
  Single strand DNA oligonucleotide
aggtttcacttgcatgcattataaaaaagcattgcttatcaatttgttgcaacgaacaggagatgtcta

SEQ ID NO: 33 - DNA, 69 bp, Artificial sequence
  Single strand DNA oligonucleotide
tagacatctcctgttcgttgcaacaaattgataaggaatgcttttttataatgcatgcaagtgaaacct

SEQ ID NO: 34 - DNA, 69 bp, Artificial sequence
  Single strand DNA oligonucleotide
aggtttcacttgcatgcattataaaaaagcattccttatcaatttgttgcaacgaacaggagatgtcta

SEQ ID NO: 35 - DNA, 69 bp, Artificial sequence
  Single strand DNA oligonucleotide
tagacatctcctgttcgttgcaacaaattgttaagcaatgcttttttataatgcatgcaagtgaaacct

SEQ ID NO: 36 - DNA, 69 bp, Artificial sequence
  Single strand DNA oligonucleotide
aggtttcacttgcatgcattataaaaaagcattgcttaacaatttgttgcaacgaacaggagatgtcta

SEQ ID NO: 37 - DNA, 69 bp, Artificial sequence
  Single strand DNA oligonucleotide
tagacatctcctgttcgttgcaacaaattcataagcaatgcttttttataatgcatgcaagtgaaacct

SEQ ID NO: 38 - DNA, 69 bp, Artificial sequence
  Single strand DNA oligonucleotide
aggtttcacttgcatgcattataaaaaagcattgcttatgaatttgttgcaacgaacaggagatgtcta

SEQ ID NO: 39 - DNA, 69 bp, Artificial sequence
  Single strand DNA oligonucleotide
tagacatctcctgttcgttgcaacaaatagataagcaatgcttttttataatgcatgcaagtgaaacct

SEQ ID NO: 40 - DNA, 69 bp, Artificial sequence
  Single strand DNA oligonucleotide
aggtttcacttgcatgcattataaaaaagcattgcttatctatttgttgcaacgaacaggagatgtcta

SEQ ID NO: 41 - DNA, 69 bp, Artificial sequence
  Single strand DNA oligonucleotide
tagacatctcctgttcgttgcaacatattgataagcaatgcttttttataatgcatgcaagtgaaacct

SEQ ID NO: 42 - DNA, 69 bp, Artificial sequence
  Single strand DNA oligonucleotide
aggtttcacttgcatgcattataaaaaagcattgcttatcaatatgttgcaacgaacaggagatgtcta

SEQ ID NO: 43 - DNA, 6 bp, Artificial sequence
  Telomer basic repeat unit
ttaggg

SEQ ID NO: 44 - DNA, 50 bp, Artificial sequence
  Single strand DNA oligonucleotide
tgccccaggacacaaagcttgtattacagacttatgtttaaagaggcatt

SEQ ID NO: 45 - DNA, 55 bp, Artificial sequence; identical to SEQ ID NO: 6
  Single strand DNA oligonucleotide